(19) United States
(12) Patent Application Publication
(10) Pub. No.: US 2023/0158077 A1
(43) Pub. Date: May 25, 2023

(54) METHODS AND COMPOSITIONS TO CONTROL CELLULAR EXPRESSION

(71) Applicant: UNIVERSITY OF WYOMING, Laramie, WY (US)

(72) Inventors: Benjamin NOREN, Laramie, WY (US); John OAKEY, Laramie, WY (US); Zhongliang JIANG, Laramie, WY (US); Kun JIANG, Laramie, WY (US)

(21) Appl. No.: 17/990,195

(22) Filed: Nov. 18, 2022

Related U.S. Application Data
(60) Provisional application No. 63/281,937, filed on Nov. 22, 2021.

(51) Int. Cl. A61K 35/28; C12N 5/0775; C12N 5/00; A61K 9/50

(52) U.S. Cl. CPC A61K 35/28; C12N 5/0663; C12N 5/0012; A61K 9/5031; C12N 2500/44; C12N 2533/30; C12N 2539/00; C12N 2537/10

(57) ABSTRACT

Embodiments of the present disclosure generally relate to methods and compositions for controlling cellular expression. More specifically, embodiments described herein relate to hydrogel-encapsulated/dispersed cells, methods of forming hydrogel-encapsulated/dispersed cells, and methods of using hydrogel-encapsulated/dispersed cells for controlling production of, for example, secretomes. In an embodiment, a composition for controlling production of secretomes is provided. The composition includes, a hydrogel comprising, in polymerized form, one or more photoreactive monomers and a thiol linker, wherein at least one of the one or more photoreactive monomers comprises a methylene functional group; and one or more cells dispersed or encapsulated within the hydrogel.

### CROSS-REFERENCES TO RELATED APPLICATIONS

This application claims priority to U.S. Provisional Patent Application No. 63/281,937, filed Nov. 22, 2021, which is incorporated herein by reference in its entirety.

### BACKGROUND

### Field

Embodiments of the present disclosure generally relate to methods and compositions for controlling cellular expression. More specifically, embodiments described herein relate to hydrogel-encapsulated/dispersed cells, methods of forming hydrogel-encapsulated/dispersed cells, and methods of using hydrogel-encapsulated/dispersed cells for controlling production of, for example, secretomes.

### Description of the Related Art

Secretomes are proteins, such as cytokines and growth factors, expressed by organisms and secreted into the extracellular space in response to, for example, injury or trauma. As an example, trophic factors including transforming growth factor-β(TGF-β), fibroblast growth factor-2 (FGF-2), and vascular endothelial growth factor-α (VEGF-α) are secreted from stem cells and can promote the regeneration of damaged cartilage via activation of molecular signaling pathways for the proliferation and regeneration of chondrocytes. As a result, stem cell-based therapies are promising strategies to repair and regenerate damaged tissues. While clinical cases regarding in vivo stem cell injection have been reported, the lack of both physical and biochemical supportive scaffold materials has constrained the functionality of injected stem cells and their fate in the inhospitable environment around damaged tissues.

Early attempts at using macroscale cellularized hydrogels have minimized these issues by isolating cells from external hazards, but their application is ultimately limited by, for example, the expense of surgical implantation, the lack of uniform, standardized cell encapsulation/dispersion procedures, and the inducement of undesirable cell behavior. Controlling the release of hydrogel-encapsulated/dispersed cells from the hydrogels also remains a challenge.

There is a need for improved methods of encapsulating/dispersing cells in hydrogels that overcome one or more deficiencies in the art. There is also a need for hydrogel-encapsulated/dispersed cells with consistent and tunable properties in order to, for example, control the production of secretomes and control the release of cells from the hydrogels.

### SUMMARY

Embodiments of the present disclosure generally relate to methods and compositions for controlling cellular expression. More specifically, embodiments described herein relate to hydrogel-encapsulated/dispersed cells, methods of forming hydrogel-encapsulated/dispersed cells, and methods of using hydrogel-encapsulated/dispersed cells for controlling production of, for example, secretomes. The inventors have found that encapsulating/dispersing cells in hydrogels can enable control over, for example, cellular expression and behavior. The inventors also found that the hydrogel properties and dimensions can be controlled and such control can enable different release profiles of cells from the hydrogels, thereby also affecting cellular expression and behavior. The hydrogel-encapsulated/dispersed cells can be useful for tissue regeneration and other therapeutic applications.

In an embodiment, a composition for controlling production of secretomes is provided. The composition includes, a hydrogel comprising, in polymerized form, one or more photoreactive monomers and a thiol linker, wherein at least one of the one or more photoreactive monomers comprises a methylene functional group, an acid functional group, or combinations thereof; and one or more cells dispersed or encapsulated within the hydrogel.

Implementations can include one or more of the following. The composition can be characterized as having less than about 20% of the cells released from the hydrogel within 48 hours after encapsulation or dispersion within the hydrogel. The diameter of the hydrogel can be from about 100 μm to about 180 μm. The composition can be characterized as having about 80% or more of the cells remain viable for 300 hours or more after dispersion or encapsulation within the hydrogel. The one or more photoreactive monomers can include polyethylene glycol norbornene, polyethylene glycol diacrylate, polylactic acid, derivatives thereof, or a combination thereof. The thiol linker can have a molecular weight of about 10 kDa or less. The thiol linker can have a molecular weight of about 5 kDa or less.

In another embodiment, a composition for controlling cytokine production is provided. The composition includes, a hydrogel comprising, in polymerized form, one or more photoreactive monomers and a thiol linker, wherein at least one of the one or more photoreactive monomers comprises a methylene functional group; and one or more cells dispersed in or encapsulated within the hydrogel. Implementations can include one or more of the following. The composition can be characterized as having less than about 20% of the cells released from the hydrogel within 48 hours after encapsulation or dispersion within the hydrogel. The diameter of the hydrogel can be from about 100 μm to about 180 μm. The composition can be characterized as having about 80% or more of the cells remain viable for 300 hours or more after dispersion or encapsulation within the hydrogel. The one or more photoreactive monomers can include polyethylene glycol norbornene, polyethylene glycol diacrylate, polylactic acid, derivatives thereof, or a combination thereof. The thiol linker can have a molecular weight of about 10 kDa or less. The thiol linker can have a molecular weight of about 5 kDa or less.

In another embodiment, a controlled release composition is provided. The composition includes, a hydrogel comprising, in polymerized form, one or more photoreactive monomers and a thiol linker, wherein at least one of the one or more photoreactive monomers comprises a methylene functional group; and one or more cells dispersed in or encapsulated within the hydrogel. Implementations can include one or more of the following. The composition can be characterized as having less than about 20% of the cells released from the hydrogel within 48 hours after encapsulation or dispersion within the hydrogel. The diameter of the hydrogel can be from about 100 μm to about 180 μm. The composition can be characterized as having about 80% or more of the cells remain viable for 300 hours or more after dispersion or encapsulation within the hydrogel. The one or more photoreactive monomers can include polyethylene glycol norbornene, polyethylene glycol diacrylate, polylactic acid, derivatives thereof, or a combination thereof. The thiol linker can have a molecular weight of about 10 kDa or less. The thiol linker can have a molecular weight of about 5 kDa or less.

In another embodiment, a method of forming a composition for controlling production of secretomes is provided. The method includes introducing a cell to one or more photoreactive monomers and a thiol linker in a microfluidic device to form a reaction mixture; and polymerizing the reaction mixture by exposure to ultraviolet light, under polymerization conditions, to form the composition for controlling production of secretomes.

Implementations can include one or more of the following. The polymerization conditions can include a duration of exposure to ultraviolet light from about 1 millisecond to about 60 seconds; an energy density of the ultraviolet light from about 1 mW/cm² to about 10,000 mW/cm²; a pH of the reaction mixture from about 5 to about 9; or a combination thereof. The composition can be in the form of cells dispersed or encapsulated within a hydrogel. The hydrogel can be formed from the one or more photoreactive monomers and the thiol linker. The one or more photoreactive monomers can include polyethylene glycol norbornene, polyethylene glycol diacrylate, polylactic acid, derivatives thereof, or a combination thereof. The thiol linker can have a molecular weight of about 5 kDa or less. The one or more photoreactive monomers can include polyethylene glycol norbornene. The diameter of the hydrogel can be from about 100 μm to about 180 μm.

In another embodiment, a method for forming a therapeutic dose of a composition for controlling production of secretomes is provided. The method includes introducing a cell to one or more components to form a reaction mixture, and polymerizing the reaction mixture, under polymerization conditions, to form a cell dispersed or encapsulated within a hydrogel. The method further includes purifying, under purification conditions, the cell dispersed and/or encapsulated in the hydrogel to form a purified hydrogel-encapsulated cell or hydrogel-dispersed cell; and suspending the purified hydrogel-encapsulated cell or hydrogel-dispersed cell in a formulation to form the therapeutic dose of the composition.

Implementations can include one or more of the following. The hydrogel can include, in polymerized form, one or more photoreactive monomers and a thiol linker. The therapeutic dose of the composition formed by the method can include a composition described herein.

In another embodiment, a method for controlling production of secretomes is provided. The method includes administering a therapeutic dose of a composition for controlling production of secretomes to a human or animal, the therapeutic dose of the composition comprising a composition described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1A is a schematic of an example device for forming a hydrogel-encapsulated/dispersed cell according to at least one embodiment of the present disclosure.

FIG. 1B is a schematic of an example device for forming a hydrogel-encapsulated/dispersed cell according to at least one embodiment of the present disclosure.

FIG. 1C is a flowchart showing selected operations of an example process for forming hydrogel-encapsulated/dispersed cells according to at least one embodiment of the present disclosure.

FIG. 2 shows exemplary images of monodisperse droplets prior to polymerization according to at least one embodiment of the present disclosure (Scale bar=200 micron (μm)).

FIG. 3A is exemplary data showing microparticle diameters of example hydrogel-encapsulated/dispersed cells made from a 1.5 kDa or a 3.5 kDa polyethylene glycol (PEG)-dithiol linker according to at least one embodiment of the present disclosure.

FIG. 3B is exemplary data showing the swelling of example microparticles over time according to at least one embodiment of the present disclosure.

FIG. 4A is an exemplary image of encapsulated mesenchymal stem cells (MSCs) using a 4× objective lens according to at least one embodiment of the present disclosure.

FIG. 4B shows exemplary images of encapsulated MSCs using a 20× objective lens according to at least one embodiment of the present disclosure.

FIG. 4C is exemplary data showing cell viability of example hydrogel-encapsulated/dispersed cells made from a 1.5 kDa PEG-dithiol linker or a 3.5 kDa PEG-dithiol linker according to at least one embodiment of the present disclosure.

FIG. 5A shows exemplary data for cellular release properties of example hydrogel-encapsulated/dispersed cells made from a 1.5 kDa PEG-dithiol linker or a 3.5 kDa PEG-dithiol linker according to at least one embodiment of the present disclosure.

FIG. 5B shows exemplary images illustrating the proliferation of released cells over time according to at least one embodiment of the present disclosure.

FIG. 6A shows exemplary data for the gene expression of fibroblast growth factor 2 (FGF-2) of example hydrogel-encapsulated/dispersed cells according to at least one embodiment of the present disclosure.

FIG. 6B shows exemplary data for the gene expression of transforming growth factor beta (TGF-β) of example hydrogel-encapsulated/dispersed cells according to at least one embodiment of the present disclosure.

FIG. 6C shows exemplary data for the gene expression of vascular endothelial growth factor alpha (VEGF-α) of example hydrogel-encapsulated/dispersed cells according to at least one embodiment of the present disclosure.

FIGS. 7A-7C shows exemplary data for the gene expression of FGF-2 of example mixed hydrogel-encapsulated/dispersed cells (mixture of polyethylene glycol norbornene (PEGNB) and polyethylene glycol diacrylate (PEGDA)) according to at least one embodiment of the present disclosure.

FIGS. 7D-7F shows exemplary data for the gene expression of TGF-β of example mixed PEGNB/PEGDA hydrogel-encapsulated/dispersed cells according to at least one embodiment of the present disclosure.

FIGS. 7G-7I shows exemplary data for the gene expression of VEGF-α of example mixed PEGNB/PEGDA hydrogel-encapsulated/dispersed cells according to at least one embodiment of the present disclosure.

FIG. 8A shows exemplary data for the percent conversion of thiol groups as a function of exposure time to ultraviolet (UV) light in an example polymerization reaction of PEGNB and PEG-dithiol according to at least one embodiment of the present disclosure.

FIG. 8B shows exemplary data for the percent conversion of norbornene groups as a function of UV exposure time in an example polymerization reaction of PEGNB and PEG-dithiol according to at least one embodiment of the present disclosure.

FIG. 8C shows exemplary images (bright-field images in the top panel and fluorescent images in the bottom panel) of an in situ photopatterning of PEGNB posts within a straight microfluidic channel according to at least one embodiment of the present disclosure.

FIG. 8D shows exemplary data for the diameter of formed posts as a function of exposure time according to at least one embodiment of the present disclosure.

FIG. 8E shows exemplary data for the fluorescent intensity of formed posts as a function of exposure time according to at least one embodiment of the present disclosure.

FIG. 9A is exemplary data for gelation efficiency of example hydrogels, shown as elastic modulus as a function of UV exposure time, according to at least one embodiment of the present disclosure.

FIG. 9B is exemplary data for gelation efficiency of example hydrogels, shown as incorporated polymer percentage as a function of UV exposure time, according to at least one embodiment of the present disclosure.

FIG. 9C is elemental analysis of example hydrogels shown as the percentage of nitrogen (N), carbon (C), hydrogen (H), and sulfur (S) within the hydrogels according to at least one embodiment of the present disclosure.

FIG. 10 shows exemplary scanning electron microscope (SEM) images of example hydrogels made with different PEG-dithiol linkers according to at least one embodiment of the present disclosure (presence of carbon, oxygen, and sulfur in the hydrogel is indicated as red, green, and blue, respectively).

FIG. 11A shows exemplary data for swelling ratio of example hydrogels with varying linker compositions over the course of 14 days according to at least one embodiment of the present disclosure.

FIG. 11B shows exemplary data for calculated theoretical mesh size of example hydrogels over the course of 14 days according to at least one embodiment of the present disclosure.

FIG. 11C shows exemplary data for the gelation efficiency of 1.5 kDa, 3.5 kDa, or 5 kDa PEG-dithiol linkers as a function of incorporated polymer percentage over a variety of exposure times according to at least one embodiment of the present disclosure.

FIG. 11D shows exemplary data for the gelation efficiency of 1.5 kDa, 3.5 kDa, or 5 kDa PEG-dithiol linkers as a function of elastic modulus over a variety of exposure times according to at least one embodiment of the present disclosure.

FIG. 11E shows exemplary data for the equilibrium swelling ratio of polymerized PEGNB hydrogels with 1.5 kDa, 3.5 kDa, or 5 kDa PEG-dithiol linkers according to at least one embodiment of the present disclosure.

FIG. 11F shows exemplary data for the calculated theoretical mesh size of polymerized PEGNB hydrogels with 1.5 kDa, 3.5 kDa, or 5 kDa PEG-dithiol linkers according to at least one embodiment of the present disclosure.

FIG. 12A is exemplary data showing mechanical properties of example hydrogels with varying linker compositions on day 1 of incubation according to at least one embodiment of the present disclosure.

FIG. 12B is exemplary data showing mechanical properties of example hydrogels with varying linker compositions on day 63 of incubation according to at least one embodiment of the present disclosure.

FIG. 12C is exemplary data showing the elastic modulus of example hydrogels with varying linker compositions over 63 days of incubation according to at least one embodiment of the present disclosure.

Figures included herein illustrate various embodiments of the disclosure. It is contemplated that elements and features of one embodiment may be beneficially incorporated in other embodiments without further recitation.

### DETAILED DESCRIPTION

Embodiments of the present disclosure generally relate to methods and compositions for controlling cellular expression. More specifically, embodiments described herein relate to hydrogel-encapsulated/dispersed cells, methods of forming hydrogel-encapsulated/dispersed cells, and methods of using hydrogel-encapsulated/dispersed cells. Compositions comprising hydrogel-encapsulated/dispersed cells can be used for controlling the production of secretomes, such as cytokines, exosomes, vesicles, and other cellular factors, in applications such as tissue regeneration and other therapies.

As described herein, the hydrogels that encapsulate, disperse, or otherwise hold cells, can serve to, for example, enhance survival of the cells, improve retention of the cells, control delivery of the cells, and control gene expression of therapeutic cells. As further described herein, approaches to material and chemical engineering of the microenvironment provided by the hydrogel enables these and other efficacious features. In addition, as opposed to conventional therapies, the hydrogel-encapsulated/dispersed cells can be injected in a minimally invasive way.

In some embodiments, methods of forming hydrogel-encapsulated/dispersed cells include forming droplets having cells, polymerizable species, and a fluorocarbon oil in a microfluidic device. The droplets are then exposed to UV light which polymerizes the polymerizable species into a crosslinked hydrogel network encapsulating/dispersing the cells. In at least one embodiment, the hydrogel-encapsulated/dispersed cells can then be isolated and re-suspended for use in, for example, therapeutic applications including injection and topical administration.

Cell-based therapies hold great promise as therapeutic treatments with high efficacy and longevity as compared to conventional drug-delivery strategies. Among therapeutic cell candidates, intense focus has been on stem cells because of their unique pluripotency and multi-functionality to differentiate into and/or promote the regeneration of desired cell types. For example, stem cells have been favorably induced to differentiate into a variety of cell types for the repair of cartilage, liver, pancreas, skin, and nervous tissue. Notably, cartilage damage is extremely difficult to cure due to its limited capability of self-recovery, which primarily arises from its avascular and aneural nature. However, unique trophic factors secreted from stem cells including transforming growth factor-β (TGF-β), fibroblast growth factor-2 (FGF-2), and vascular endothelial growth factor-α (VEGF-α) can promote the regeneration of damaged cartilage via activation of molecular signaling pathways for proliferation and regeneration of chondrocytes. TGF-β signaling plays a role in tissue regeneration or healing processes in response to injury by regulating extracellular matrix (ECM) production, favorable cell differentiation, and promoting cell viability of newly generated cells. FGF-2 improves glycosaminoglycan (GAG) synthesis and induces chondrogenesis. VEGF-α has been validated as an inducer for proliferation and differentiation for epithelial cells both in vitro and in vivo.

Even though direct stem cell implantation has shown significantly enhanced improvement for tissue regeneration, the in vivo environment, in particular around damaged tissues, where implanted stem cells are exposed to elevated oxidative stresses and accumulated inflammatory factors, is not ideal to support long-term cell survival of the stem cells. Additionally, the direct injection of cells generally results in poor cell retention within target tissues and/or poor cell viability in suspension. Early termination or poor retention of implanted stem cells jeopardize the effectiveness of conventional cell-based therapies, necessitating repeated injections or more invasive options.

Hydrogels have been developed and employed as three-dimensional scaffolds to provide seeded cells with physiological and biochemical support and prolonged residence time in vivo. Semi-permeability enables the diffusion of small molecules, and the transparency of hydrogels enables the monitoring of cellular behavior over longer-term studies. Traditional natural materials like collagen, alginate, and fibrin, all exhibit excellent cytocompatibility; however, their translational potential as hydrogels have been hindered by a lack of controlled gelation on the microscale and immunogenicity concerns in clinical trials.

As an alternative to natural materials, PEG-based photoinitiated synthetic materials have become increasingly accepted. PEG-based free radical initiated synthetic materials, therefore, have been intensively studied and successfully employed for cell encapsulation. Photolabile macromers enable spatial and temporal control over gelation, tuning of physical and biochemical properties in formed hydrogels to recapitulate dynamic developmental cues in vivo, and ECM mimicry via covalent bonding or physical entanglement. All of these capabilities have enabled PEG-based materials to be designed as cell encapsulants that recapitulate in vivo environments to study cell-cell or cell-matrix interactions. However, most studies are based upon the macroscopic encapsulation of millions of cells using modes that operate over length scales of millimeters to centimeters. Further, evaluation of these conventional scaffolds assumes homogeneity, and data interpretation is based upon averages over all encapsulated cells. This approach not only neglects interactions between individual cells, but also overlooks phenomena or heterogeneity on the diffusional length scales of biological molecules or oxygen. As a consequence, unstandardized cell encapsulation processes within macroscopic hydrogels often produce variable results with poor reproducibility. Moreover, and with respect to clinical trials, implantation of macroscopic cellularized hydrogels often require invasive surgeries, which introduce complications such as a higher risk of pathogen invasion, inconvenience, and high costs.

Miniaturization techniques have been developed to overcome issues of reproducibility in macroscale hydrogel scaffolds in order to both improve hydrogel fabrication throughput and to create injectable cell-laden microgels. The recently-developed technique of droplet-microfluidics excels at this task, particularly in comparison with alternate miniaturization techniques, including capillary lithography, continuous flow lithography (CFL), stop flow lithography (SFL), and bioprinting. However, the translational potential of conventional miniaturization schemes is ultimately constrained by, for example, low cell viability and low fabrication throughput.

Recently, studies have characterized PEGNB as a versatile cell encapsulant on both macro- and micro-length scales. Organized network structures of PEGNB can be formed with mild UV intensity, contributing to increased bioligand availability and minimized toxicity. One challenge with PEG-based hydrogels, however, is oxygen. Oxygen inhibition during photopolymerization, which is governed by polymerization kinetics and oxygen diffusivity, occurs in all photoinitiated PEG hydrogel-forming formulations, but is exacerbated in diminishing volumes due to increased surface-to-volume ratio. As a result, on-chip fabrication of hydrogel particles remain difficult. Reactive oxygen species (ROS) produced as byproducts of oxygen inhibition can further reduce cell viability by rapidly damaging essential cellular macromolecules, including DNA and proteins. While photoinitiators within hydrogel-forming solutions are susceptible to oxygen inhibition due to radical scavenging, PEGNB mitigates the deleterious effects of ROS. Such mitigation is presumably due to the deactivation or consumption of ROS during crosslinking, though the mechanism of this effect is not fully understood. This unique feature of PEGNB allows miniaturizing cellularized microparticles with comparative cell viability to macroscale hydrogels within an environment that permits rapid oxygen diffusion.

While the step-growth crosslinking of PEGNB macromers is not itself sensitive to oxygen inhibition, gel formation generally takes longer under cytocompatible conditions, and it is even more exaggerated when linker neutralization induced gelation defects occurs in thiol-ene hydrogels. The mismatch between high droplet production rates and relatively slow polymerization rates has presented challenges to achieving live cell encapsulation within PEGNB microparticles on-chip. In addition, PEGNB hydrogels formed with linkers of various lengths display different mechanical characteristics, but limited effort has been made to investigate how these changes in physical characteristics affect the behavior of encapsulated/dispersed cells.

Embodiments described herein can enable high-throughput generation of hydrogel microparticles by utilizing photoreactive groups chemically attached to PEG hydrogels to form a material which, upon exposure to specific wavelengths of light, react to form a crosslinked hydrogel network of customizable physical and chemical properties. Embodiments utilize this technique to create tunable, biocompatible microenvironments suitable for encapsulation/dispersion of living cells in sufficient quantities and rapid enough timespans to enable their scaled production. The formulation of encapsulated material enables material and chemical engineering of the microenvironment to control the expression of cellular factors secretomes, such as cytokines, exosomes, vesicles etc. The control afforded by the compositions and methods described herein enables an array of applications including, for example, production of targeted biological agents in a bioreactor configuration, and customized panels of biomolecules excreted in situ to enable improved regenerative therapies with targeted and customizable secretome (for example, cytokine) production better than the state-of-the-art. Further, the compositions and methods described herein can be useful for speed-healing applications, as well as provide superior tissue regeneration outcomes relative to conventional methods and compositions.

As described herein, and in some embodiments, monodisperse PEGNB droplets are produced in a high-throughput manner and photopolymerized into microparticles on-chip. The droplets can travel along a short channel or a serpentine channel to extend photopolymerization time. Processing parameters described herein enable high post-encapsulation cell viability, which can be maintained at, for example, above about 90% over the course of about 14 days. Additionally, crosslinking efficiency-induced variation in mesh size arising from the use of two crosslinkers can result in different cell release profiles. In some examples, and to the understand cellular response to the hydrogel-encapsulation/dispersion, the functionality of encapsulated MSCs characterized transcriptionally via the expression of target genes immediately after encapsulation/dispersion and after 14 days of culture. Gene expression immediately post-encapsulation was comparable to MSCs harvested from monolayer cultures, and was the similar regardless of micro- or macro-encapsulation. Over time in culture, however, MSCs sense differences in scaffold material properties and/or dimensions and demonstrate variable trophic factor expression levels. These results indicate MSC-laden, injectable microparticles (made from, for example, PEGNB or PEGNB-PEGDA) can be tailored to applications for tissue engineering and regenerative medicines via the understanding and manipulation of desired cellular response.

Although embodiments described herein are discussed with reference to hydrogel encapsulation of cells, it is contemplated that the cells can be dispersed, retained, or otherwise held in the hydrogels.

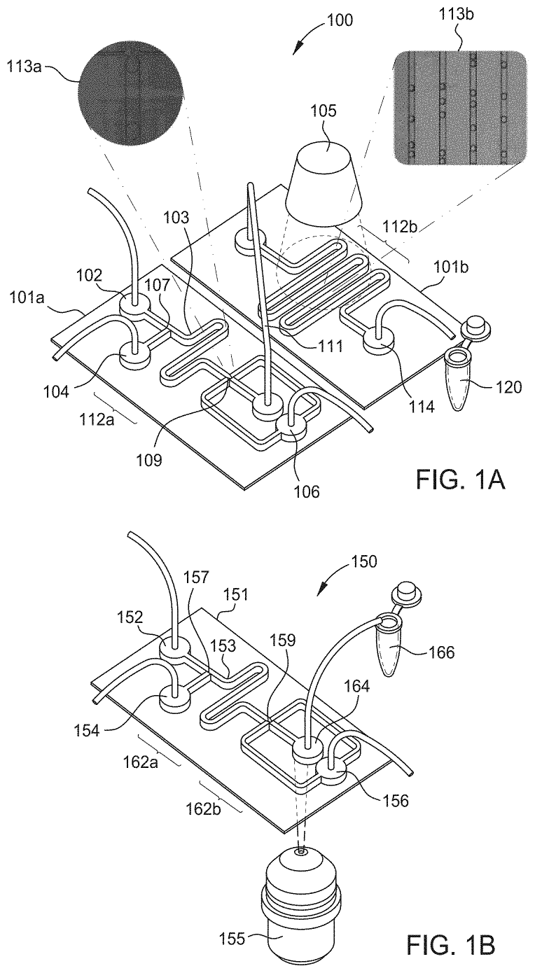

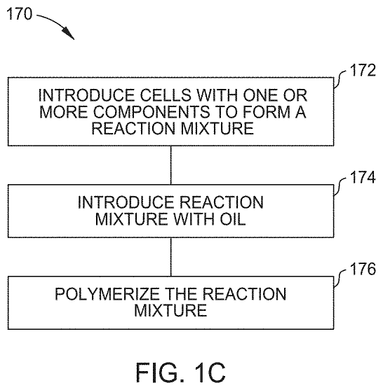

Embodiments described herein generally relate to devices for forming hydrogel-encapsulated/dispersed cells. FIGS. 1A and 1B are schematics of an example device for forming hydrogel-encapsulated/dispersed cells according to some embodiments of the present disclosure. Such hydrogel-encapsulated/dispersed cells produced can be in the form of microparticles. Device 100 (FIG. 1A) and device 150 (FIG. 1B) can be used for continuous production of hydrogel-encapsulated/dispersed cells.

In general, the choice of device depends on, for example, material properties a user desires for the formed hydrogel as well as the polymerization reaction kinetics to form the hydrogel. For example, device 100 can be used with thiol linkers and/or polymerization monomers that exhibit relatively slower rates of polymerization. As another example, relatively faster polymerizing linkers and/or monomers can be polymerized in device 150 of FIG. 1B.

With reference to FIG. 1A, device 100 includes a first microfluidic device 101a and a second microfluidic device 101b. The first microfluidic device 101a and the second microfluidic device 101b share a fluidic channel 103 extending from the first microfluidic device 101a and the second microfluidic device 101b. Although the device 100 is shown to include two microfluidic devices—first microfluidic device 101a and second microfluidic device101b,—it is contemplated that only one microfluidic device can be used or more than two microfluidic devices can be utilized. In at least one embodiment, the fluidic channel 103 has a diameter of micrometers (μm) to millimeters. For example, the fluidic channel 103 has a diameter from about 1 μm to about 2 mm and/or a depth of about 1 μm to about 2 mm. One or more portions of the fluidic channel 103 is in the form of loops, discussed below. The fluidic channel 103 includes a mixing area 112a where the hydrogel forming solution (discussed below) can be mixed with cells, and a polymerization area 112b where monomers of the hydrogel forming solution polymerize to form hydrogels that encapsulate and/or disperse the cells. Portions of the fluidic channel 103 are in the form of loops. The loops enable control over, for example, the kinetics of mixing, the kinetics of polymerization, exposure time for polymerization, the gelation of the hydrogels. That is, the loops enable uniform processing of microparticles. Other morphologies or shapes besides, or in addition to, loops are contemplated to enable processing of the microparticles, such as spirals. That is, any suitable shape that extends the length of the fluidic channel 103 in the area under which UV light is exposed would have the same or similar effect of controlling the exposure time so that the desired crosslinking can be achieved on a microfluidic chip with high throughput droplet production capabilities.

The microfluidic device 101a has an opening 102 for introducing a hydrogel forming solution to the fluidic channel 103. The hydrogel forming solution includes photoinitiators, reaction components and/or photoreactive monomers (for example, PEG-dithiol linker, PEGNB, PEGDA, PLA, etc.). The first microfluidic device 101a includes another opening 104 for introducing cells, such as MSCs, suspended in a suspension fluid, and an opening 106 to introduce a fluorocarbon oil. Openings 102, 104, and 106 are coupled to the fluidic channel 103. Although three openings are described, more or less openings can be used to introduce the suspension fluid, cells, photoreactive monomers, photoinitiators, reaction components, fluorocarbon oil, etc. to the first microfluidic device 101a. The fluidic channel 103 includes a junction 107 where the hydrogel forming solution and the suspended cells meet and travel into the loops of the mixing area 112a of the fluidic channel 103. As shown, tubings are coupled to the individual openings 102, 104, and 106 to allow introduction of the components to the fluidic channel 103. However, it is contemplated that introduction of components can be performed in other manners, such as direct connecting Leuer lock type devices, snap-together microfluidic assemblies, syringe-like devices, without departing from the scope of the present disclosure.

The fluidic channel 103 includes another junction 109 where the fluorocarbon oil pinches off the hydrogel forming solution and the suspended cells to form droplets. The droplets include, but are not limited to, cells, suspension fluid photoreactive monomers, photoinitiators, reaction components, and/or fluorocarbon oil, as well as other materials. Inset 113a is an image of the fluidic channel 103 at junction 109. After the droplets are formed, the droplets travel along the fluidic channel 103 through a travel path 111 towards the polymerization area 112b of second microfluidic device 101b. At the polymerization area 112b, monomers and/or reaction components polymerize to, for example, a hydrogel, that encapsulates, disperses, retains, or otherwise holds a cell(s). As shown, the fluidic channel 103 of the polymerization area 112b includes a suitable number of loops to enable, for example, sufficient polymerization of the monomers and other reaction components as well as sufficient gelation of the hydrogels.

The fluidic channel 103 is coupled to a polymerization control device 105. The polymerization control device 105 is configured to cause a polymerization reaction when the desired materials are within the polymerization area 112. The polymerization control device 105 can include a UV-light source(s) that polymerizes the monomers to form the hydrogel. Coupling of the polymerization control device 105 can take multiple forms. For example, the microfluidic device 101b is placed on top of, below, or otherwise adjacent to, a UV-light source(s), as shown in FIG. 1A. Here, the UV light source, such as a UV lamp, is located in a stand-alone machine outside of the microfluidic device 101b, and as such, the fluidic channel 103 is optically coupled to the polymerization control device 105. As another example, the fluidic channel 103 is coupled to the polymerization control device 105. Inset 113b is an image of the droplets and polymerized hydrogels within the fluidic channel 103 of the polymerization area 112. After polymerization, the cell-laden microparticles (for example, the hydrogel-encapsulated/dispersed cells) move toward the fluidic channel exit 114 where the cell-laden microparticles can be collected via any suitable collection unit 120, for example, flask, centrifuge tube, reservoir, vessel, or the like.

Other materials (byproducts, suspension fluid, unreacted materials, etc.) exit the fluidic channel exit 114 along with the hydrogel-encapsulated/dispersed cells. Accordingly, and in some embodiments, the hydrogel-encapsulated cells are purified, or otherwise isolated, from the other materials exiting the device 100. Movement of the various materials (for example, suspension fluid, and cells, photoreactive monomers, and/or reaction components) from the one or more openings 102, 104, and 106 to the fluidic channel exit 114 is controlled by, for example, capillary action, laminar flow, temperature, a pumping mechanism (for example, a piezoelectric pump), electrodes, and the like. Such elements controlling the movement can be placed at opposing ends of or along various regions along a length of the fluidic channel 103.

As described above, FIG. 1B is a schematic of an example of a device 150 for forming hydrogel-encapsulated/dispersed cells according to at least one embodiment of the present disclosure. Device 150 includes a microfluidic device 151. Although the device 150 is shown to include one microfluidic device 151, it is contemplated that more than one microfluidic device can be used. The microfluidic device 151 includes a fluidic channel 153. In at least one embodiment, the fluidic channel 153 has a diameter of micrometers (μm) to millimeters. For example, the fluidic channel 153 has a diameter from about 1 μm to about 2 mm and/or a depth of about 1 μm to about 2 mm. One or more portions of the fluidic channel 153 is in the form of loops, discussed below. The fluidic channel 153 includes a mixing area 162a where the hydrogel forming solution (discussed below) can be mixed with cells, and a polymerization area 162b where monomers of the hydrogel forming solution polymerize to form hydrogels that encapsulate and/or disperse the cells. One or more portions of the fluidic channel 153 are in the form of loops as discussed above.

The microfluidic device 151 has an opening 152 for introducing a hydrogel forming solution to the fluidic channel 153. The hydrogel forming solution includes photoinitiators, reaction components, photoreactive monomers (for example, PEG-dithiol linker, PEGNB, PEGDA, PLA, among others), or combinations thereof. The microfluidic device 151 includes another opening 154 for introducing cells, such as MSCs, suspended in a suspension fluid, and an opening 156 to introduce a fluorocarbon oil. Openings 152, 154, and 156 are coupled to the fluidic channel 153. Although three openings are described, more or less openings can be used to introduce the suspension fluid, cells, photoreactive monomers, photoinitiators, reaction components, fluorocarbon oil, etc. to the microfluidic device 151. The fluidic channel 153 includes a junction 157 where the hydrogel forming solution and the suspended cells meet and travel into the loops of the mixing area 162a of the fluidic channel 153. As shown, tubings are coupled to the individual openings 152, 154, and 156 to allow introduction of the components to the fluidic channel 153. However, it is contemplated that introduction of components can be performed in other manners, such as such as direct connecting Leuer lock type devices, snap-together microfluidic assemblies, syringe-like devices, without departing from the scope of the present disclosure.

The fluidic channel 153 includes another junction 159 where the fluorocarbon oil pinches off the hydrogel forming solution and the suspended cells to form droplets. The droplets include, but are not limited to, cells, suspension fluid photoreactive monomers, photoinitiators, reaction components, and/or fluorocarbon oil, as wells as other materials. After the droplets are formed, the droplets travel along the fluidic channel 103 towards the polymerization area 162b of the microfluidic device 151. At the polymerization area 162b, monomers and/or reaction components polymerize to, for example, a hydrogel, that encapsulates, disperses, retains, or otherwise holds a cell(s). Although not shown, the polymerization area 162b can include any suitable number of loops to enable sufficient time for polymerization and/or gelation of the hydrogels.

The fluidic channel 153 is coupled to a polymerization control device 155. The polymerization control device 155 is configured to cause a polymerization reaction when the desired materials are within the polymerization area 162b. The polymerization control device 155 can include a UV-light source(s) that polymerizes the monomers to form the hydrogel. Coupling of the polymerization control device 155 can take multiple forms. For example, the microfluidic device 151 is placed on top of, below, or otherwise adjacent to, a UV-light source(s), as shown in FIG. 1B. Here, the UV light source, such as a UV lamp, is located in a stand-alone machine outside of the microfluidic device 151, and as such, the fluidic channel 153 is optically coupled to the polymerization control device 155. As another example, the fluidic channel 153 is coupled to the polymerization control device 155. After polymerization, the cell-laden microparticles (for example, the hydrogel-encapsulated/dispersed cells) move toward the fluidic channel exit 164 where the cell-laden microparticles can be collected via any suitable collection unit 166, for example, flask, centrifuge tube, reservoir, vessel, or the like.

Referring back to FIG. 1B, other materials (byproducts, suspension fluid, unreacted materials, etc.) exit the fluidic channel exit 164 along with the hydrogel-encapsulated/dispersed cells. Accordingly, and in some embodiments, the hydrogel-encapsulated cells are purified, or otherwise isolated, from the other materials exiting the device 150. Movement of the various materials (for example, suspension fluid, and cells, photoreactive monomers, and/or reaction components, etc.) from the one or more openings 152, 154, and 156 to the fluidic channel exit 164 is controlled by, for example, capillary action, laminar flow, temperature, a pumping mechanism (for example, a piezoelectric pump), electrodes, and the like. Such elements controlling the movement can be placed at opposing ends of or along various regions along a length of the fluidic channel 153.

Cells suspended, dispersed, encapsulated, or otherwise held within the hydrogel include, but are not limited to, mesenchymal stem cells (MSCs), mesenchymal stromal cells, perinatal cells, fat derived stem cells, bone marrow aspirate concentrate, and beta cells. It is contemplated that any other cell can also be suspended, dispersed, encapsulated, or otherwise held in the hydrogel particles.

Embodiments of the present disclosure also generally relate to compositions for controlling production of, for example, secretomes. The compositions include a hydrogel and one or more cells dispersed or encapsulated within the hydrogel. The hydrogel includes, in polymerized form, one or more photoreactive monomers and a thiol linker. The hydrogel can be formed from a reaction mixture that includes one or more photoreactive monomers, one or more linkers, one or more photoinitiators, other component(s), solvent(s), or combinations thereof. Other component(s) can include one or more cell adhesion peptides, among others. This reaction mixture is interchangeably referred to as a hydrogel forming solution unless the context indicates otherwise.

As used herein, a “composition” can include component(s) of the composition, reaction product(s) of two or more components of the composition, a remainder balance of remaining starting component(s), or combinations thereof. Compositions of the present disclosure can be prepared by any suitable mixing process.

The photoreactive monomers contain photoreactive functional groups. Upon irradiation, the monomers form a hydrogel. The one or more photoreactive monomers used to form the hydrogel can include a methylene functional group, an acid functional group, or combinations thereof. The one or more photoreactive monomers can include photoreactive groups chemically attached to, e.g., polyethylene glycol (PEG). Illustrative, but non-limiting, examples of photoreactive functional groups include alkenes, thiols, acids, or combinations thereof. Upon irradiation, the photoreactive monomers (with or without co-reactants, such as linkers described below) can react to form a hydrogel.

Non-limiting examples of photoreactive monomers include, but are not limited to, polyethylene glycol norbornene (PEGNB), polyethylene glycol diacrylate (PEGDA), PEG methacrylate, polyethylene glycol di-photodegradable acrylate (PEGdiDPA), derivatives thereof, or combinations thereof. The photoreactive monomers can be branched (e.g., ˜20 k 4-arm PEGNB and ˜40 k 8-arm PEGNB) or unbranched. Other PEG-based derivatives having varied reactive functional groups are also contemplated. The molecular weight and shape (e.g., number of arms on PEGNB) of the one or more photoreactive monomers, among other characteristics, can be changed. Changing the molecular weight and shape of the photoreactive monomers (as well as the linker) can enable the tuning of various properties of the hydrogel structure and can confer a range of traits to the system depending on the desired use and desired effect on the cells. Photoreactive monomers can also include non-PEG-based monomers such as acrylates and acids (for example, lactic acid). For example, polylactic acid (PLA) and derivatives thereof can be used. Block copolymers and triblock copolymers can also be used such as triblock PLA.

Photoreactive monomers can also include non-PEG-based monomers such as acrylates, acids (e.g., lactic acid, hyaluronic acid), gelatin, collagen, or combinations thereof. For example, polylactic acid (PLA), acrylated hyaluronic acid, gelatin methacrylate, derivatives thereof, and combinations thereof can be used. Block copolymers and triblock copolymers can also be used such as triblock PLA and PLA-PEG-PLA.

Molecular conformation of the photoreactive monomers can be varied to, for example, impart desired material properties to the hydrogel microenvironment. For example, 1-arm molecular structures to 12-arm molecular structures can be used, such as 4-arm, 8-arm, or 12-arm molecular structures, such as 4-arm PEGNB, 8-arm PEGNB, 12-arm PEGNB, or combinations thereof.

Further, the chemical properties of the hydrogel microenvironment can be modified via click chemistry through addition of thiolated agents (for example, PEGNB) or similar acrylated agents (for example, PEGNB) such as thiolated cell adhesion peptides like RGD (arginine-glycine-aspartate) or CRGDS (cystine-arginine-glycine-aspartate-serine). Mixtures of one or more photoreactive monomers, for example, a mixture of PEGNB and PEGDA) can also be used, as well as mixtures that include non-PEG-based photolabile hydrogels such as gelatin methacrylate and/or photolabile hyaluronic acid.

In at least one embodiment, the one or more photoreactive monomers comprises polyethylene glycol norbornene, polyethylene glycol diacrylate, polylactic acid, derivatives thereof, or a combination thereof.

A molecular weight of the one or more photoreactive monomers can be from about 100 Da to about 75,000 Da, such as from about 250 Da to about 50,000 Da, such as from about 5,000 Da to about 50,000 Da, such as from about 10,000 Da to about 45,000 Da, such as from about 15,000 Da to about 40,000 Da, such as from about 20,000 Da to about 35,000 Da, such as from about 25,000 Da to about 30,000 Da. Illustrative, but non-limiting, examples of the molecular weight of the photoreactive monomer are from about 250 Da to about 10,000 Da, such as from about 500 Da to about 9,000 Da, such as from about 1,000 Da to about 8,000 Da, such as from about 2,000 Da to about 7,000 Da, such as from about 3,000 Da to about 6,000 Da, such as from about 4,000 Da to about 5,000 Da. In some examples, the molecular weight of the one or more photoreactive monomers is 30,000 Da or less. In some embodiments, the molecular weight of the one or more photoreactive monomers ranges from MW₁ to MW₂ where each of MW₁ to MW₂ (in Da) is, independently, about 200, about 300, about 400, about 500, about 600, about 700, about 800, about 900, about 1,000, about 1,500, about 2,000, about 2,500, about 3,000, about 3,500, about 4,000, about 4,500, about 5,000, about 5,500, about 6,000, about 6,500, about 7,000, about 7,500, about 8,000, about 8,500, about 9,000, about 9,500, about 10,000, about 10,500, about 11,000, about 11,500, about 12,000, about 12,500, about 13,000, about 13,500, about 14,000, about 14,500, about 15,000, about 15,500, about 16,000, about 16,500, about 17,000, about 17,500, about 18,000, about 18,500, about 19,000, about 19,500, about 20,000, about 20,500, about 21,000, about 21,500, about 22,000, about 22,500, about 23,000, about 23,500, about 24,000, about 24,500, about 25,000, about 25,500, about 26,000, about 26,500, about 27,000, about 27,500, about 28,000, about 28,500, about 29,000, about 29,500, about 30,000, about 30,500, about 31,000, about 31,500, about 32,000, about 32,500, about 33,000, about 33,500, about 34,000, about 34,500, about 35,000, about 35,500, about 36,000, about 36,500, about 37,000, about 37,500, about 38,000, about 38,500, about 39,000, about 39,500, about 40,000, about 40,500, about 41,000, about 41,500, about 42,000, about 42,500, about 43,000, about 43,500, about 44,000, about 44,500, about 45,000, about 45,500, about 46,000, about 46,500, about 47,000, about 47,500, about 48,000, about 48,500, about 49,000, about 49,500, or about 50,000, as long as MW₁<MW₂. Higher or lower molecular weights of the one or more photoreactive monomers are contemplated. The molecular weight of the photoreactive monomer refers to the number average molecular weight (Mₙ). The Mₙ is the Mₙ provided by the manufacturer of the photoreactive monomer.

Suitable organic and/or aqueous solvents are utilized as a portion of the hydrogel forming solution. Such organic and/or aqueous solvents can include water, saline, phosphate buffered saline, appropriate biologically compatible liquid, or combinations thereof.

A concentration of the one or more photoreactive monomers in the hydrogel forming solution can be from about 5 wt % to about 75 wt %, such as from about 10 wt % to about 70 wt %, such as from about 15 wt % to about 65 wt %, such as from about 20 wt % to about 60 wt %, such as from about 25 wt % to about 55 wt %, such as from about 30 wt % to about 50 wt %, such as from about 35 wt % to about 45 wt %, based on a total weight percent of the components of the hydrogel forming solution (not to exceed 100 wt %). In at least one embodiment, the concentration of the one or more photoreactive monomers in the hydrogel forming solution is from about 5 wt % to about 35 wt %, such as from about 10 wt % to about 30 wt %, such as from about 15 wt % to about 25 wt %, based on the total weight percent of the components of the hydrogel forming solution (not to exceed 100 wt %). Any of the foregoing numbers can be used singly to describe an open-ended range or in combination to describe a close-ended range. Higher or lower concentrations of the one or more photoreactive monomers can be used depending on application.

The components that are subjected to polymerization can further include one or more linkers, such as a dithiol linker, such as a polyethylene glycol-dithiol (PEG-dithiol) linker, a derivative thereof, or combinations thereof. PEG-dithiol is a thiolated PEG having two thiol groups. The linker can be referred to as a thiol-containing monomer or dithiol linker unless the context indicates otherwise. When a dithiol linker is utilized, the photoreactive monomer(s) can polymerize with the thiol-containing monomer(s) via, for example, a step-growth polymerization reaction occurring between the ene portion of the monomers and the thiol of the thiol-containing monomer.

A molecular weight of the one or more linkers (for example, the PEG-dithiol linker) can be from about 500 Da to about 10,000 Da, such as from about 1,000 Da to about 9,500 Da, such as from about 1,500 Da to about 9,000 Da, such as from about 2,000 Da to about 8,500 Da, such as from about 2,500 Da to about 8,000 Da, such as from about 3,000 Da to about 7,500 Da, such as from about 3,500 Da to about 7,000 Da, such as from about 4,000 Da to about 6,500 Da, such as from about 4,500 Da to about 6,000 Da, such as from about 5,000 Da to about 5,500 Da. In some examples, the molecular weight of the linker is about 6,000 Da or less, such as from about 500 Da to about 6,000 Da, such as from about 1,000 Da to about 5,000 Da, such as from about 1,500 Da to about 4,500 Da, such as from about 2,000 Da to about 4,000 Da, such as from about 2,500 Da to about 3,500 Da. The molecular weight of the linker refers to the number average molecular weight (Mₙ). The Mₙ is the Mₙ provided by the manufacturer of the linker. Any of the foregoing numbers can be used singly to describe an open-ended range or in combination to describe a close-ended range. Higher or lower molecular weights of the one or more linkers are contemplated. Illustrative, but non-limiting, examples of PEG-dithiol linkers include ˜1.5 k PEG-dithiol, 3.5 k PEG-dithiol, and ˜5 k PEG-dithiol.

A concentration of the one or more linkers (for example, PEG-dithiol linker) in the hydrogel forming solution can be from about 1 mM to about 50 mM, such as from about 5 mM to about 45 mM, such as from about 10 mM to about 40 mM, such as from about 15 mM to about 35 mM, such as from about 20 mM to about 30 mM, based on a total molar concentration of the components of the hydrogel forming solution. Any of the foregoing numbers can be used singly to describe an open-ended range or in combination to describe a close-ended range. Higher or lower concentrations of the one or more linkers can be used depending on application.

The hydrogel forming solution can also include one or more photoinitiators. Illustrative, but non-limiting, examples of photoinitiators include lithium phenyl-2,4,6-trimethylbenzoylphosphinate (LAP) photoinitiator, 2-hydroxy-2-methyl propiophenone (for example, Irgacure™ 1173, Darocur™ 1173), and combinations thereof. The photoinitiator induces polymerization when exposed to UV light. The use of LAP, due to its robust initiation properties at low concentrations, can minimize deleterious effects on sensitive cells. The aforementioned solution suspended in an aqueous carrier is passed through a flow focusing, cross flow, or similar microfluidic channel to produce highly monodisperse, cell-laden microparticles.

A concentration of the one or more photoinitiators in the hydrogel forming solution can be from about 0.0001 wt % to about 1 wt %, such as from about 0.001 wt % to about 0.9 wt %, such as from about 0.01 wt % to about 0.5 wt %, such as from about 0.05 wt % to about 0.1 wt %, based on the total wt % of the components of the hydrogel forming solution. Any of the foregoing numbers can be used singly to describe an open-ended range or in combination to describe a close-ended range. Higher or lower concentrations of the one or more photoinitiators can be used depending on, for example, the application or desired results.

The chemical properties of the hydrogel microenvironment can be modified via click chemistry through addition of thiolated agents such as thiolated cell adhesion peptides like RGD or CRGDS. In some embodiments, the hydrogel forming solution can include one or more cell adhesion peptides, such as thiolated cell adhesion peptides, such as RGD, CRGDS, or a combination thereof. A concentration of the one or more cell adhesion peptides in the hydrogel forming solution can be from about 0.5 mM to about 10 mM, such as from about 1 mM to about 8 mM, such as from about 2 mM to about 6 mM, such as from about 3 mM to about 4 mM based on the total molar concentration of the components of the hydrogel forming solution. Any of the foregoing numbers can be used singly to describe an open-ended range or in combination to describe a close-ended range. Higher or lower concentrations are contemplated.

Cells (such as MSCs) in a suitable media such as an aqueous buffer Dulbecco's Modified Eagle's Medium (DMEM), such as phosphate buffered saline, can also introduced to the device (e.g., device 100 or device 150). The cells in media can be part of the hydrogel forming solution. A concentration of cells, such as MSCs, in the suitable media or in the hydrogel forming solution that are introduced or otherwise delivered to the device can be from about 1 cell/mL to about 1×10⁹ cells/mL, such as from about 1×10⁴ cells/mL to about 1×10⁹ cells/mL, such as from about 1×10³ cells/mL to about 1×10⁸ cells/mL or from about 1×10⁵ cells/mL to about 1×10⁸ cells/mL, such as from about 1×10⁵ cells/mL to about 1×10⁷ cells/mL, such as from about 1×10⁵ cells/mL to about 1×10⁷ cells/mL. A higher or lower concentration of cells in the suitable media or in the hydrogel forming solution can be utilized. Any of the foregoing numbers can be used singly to describe an open-ended range or in combination to describe a close-ended range. Higher or lower concentrations are contemplated.

Additional components and/or reaction mixture precursors can be used. The additional components and/or reaction mixture precursors mix and/or interact (for example, chemically and/or physically) with other components to form the hydrogel-encapsulated/dispersed cells. Such additional components and/or reaction mixture precursors can include chemicals, for example, solvents, reagents, catalysts, etc., that upon interacting with the monomers and cells, form a reaction mixture and transform the monomers and cells to the hydrogel-encapsulated cells. For example, the additional components and/or reaction mixture precursors upon interaction with the photoreactive monomers, linkers, photoinitiators, perform a polymerization operation.

Using the components described above, various formulations can be used to form the hydrogel-encapsulated/dispersed cells or compositions thereof. The formulation can be that of the hydrogel forming solution or separate solutions that are introduced to a device (for example, device 100 or device 150) or other suitable devices to form hydrogels.

A non-limiting formulation useful for the hydrogel forming solution can include (a) from about 0.1 wt % to about 40 wt %, such as from about 1 wt % to about 40 wt %, such as from about 5 wt % to about 35 wt %, such as from about 10 wt % to about 20 wt % of PEGNB (for example, 4-arm, 8-arm, or a combination thereof) ranging in molecular weight from about 500 Da to about 50,000 Da, such as from about 3,000 Da to about 50,000 Da, such as from about 5,000 Da to about 20,000 Da, such as from about 10,000 Da to about 15,000 Da; (b) from about 1 mM to about 100 mM, such as from about 5 mM to about 50 mM PEG dithiol ranging in molecular weight from about 100 Da to about 10,000 Da; (c) from about 0.0001 wt % to about 1 wt %, such as from about 0.01 wt % to about 0.1 wt % of the LAP photoinitiator; or combinations thereof. Additional components can be used as desired.

When PEGNB is utilized with another photoreactive monomer such as PEGDA, PLA, PLA-PEG-PLA, among others, a non-limiting formulation can include the aforementioned formulation with about 0.1 wt % to about 40 wt %, such as from about 1 wt % to about 40 wt %, such as from about 5 wt % to about 35 wt %, such as from about 10 wt % to about 20 wt % of the second photoreactive monomer (e.g., PEGDA, PLA, PLA-PEG-PLA, etc.) having a molecular weight from about 1,000 Da to about 30,000 Da, such as from about 5,000 Da to about 20,000 Da, such as from about 10,000 Da to about 15,000 Da. Additional components can be used as desired.

An illustrative, but non-limiting, formulation useful to form a PEGPLA/NB composite hydrogels can include: (a) about 0.1 wt % to about 40 wt %, such as from about 1 wt % to about 40 wt %, such as from about 5 wt % to about 35 wt % such as from about 10 wt % to about 20 wt % of the photoreactive monomer (for example, PLA-PEG-PLA, etc.) having a molecular weight from about 1,000 Da to about 30,000 Da, such as from about 5,000 Da to about 20,000 Da, such as from about 10,000 Da to about 15,000 Da; (b) from about 0.1 wt % to about 40 wt %, such as from about 1 wt % to about 40 wt %, such as from about 5 wt % to about 35 wt %, such as from about 10 wt % to about 20 wt % of a second photoreactive monomer (e.g., PEGNB, such as 4-arm PEGNB, 8-arm PEGNB, or a combination thereof) ranging in molecular weight from about 500 Da to about 50,000 Da, such as from about 3,000 Da to about 50,000 Da, such as from about 5,000 Da to about 20,000 Da, such as from about 10,000 Da to about 15,000 Da; (c) from about 1 mM to about 100 mM, such as from about 5 mM to about 50 mM PEG dithiol ranging in molecular weight from about 100 Da to about 10,000 Da; and/or (d) from about 0.0001 wt % to about 1 wt %, such as from about 0.01 wt % to about 0.1 wt % of the LAP photoinitiator. Additional components can be used as desired.

In one example, the hydrogel-forming solution includes about 20 wt % 4-arm 20 k PEGNB, about 20 mM 3.5 k PEG dithiol, and about 0.6 wt % LAP. Cells are then mixed with the hydrogel-forming solution on-chip in a channel. Cell-laden 10 wt % PEGNB microgels can be fabricated under a constant flow rate of 0.5 μL/min while varying oil phase flow rate to about 2 μL/min, about 5 μL/min, and about 20 μL/min. Cell adhesion peptides, such as RGD and CRGDS, can be used. For example, a hydrogel-forming solution includes ˜7 wt % 8-arm 40 k PEGNB with ˜3 mM 5 k PEG-dithiol, and ˜3 mM RGD and/or CRGDS.

In another example, PEGNB is mixed with dithiol linker, LAP, and cell-containing culture media to a final concentration of ˜10 wt % PEGNB, ˜10 mM dithiol linker, and ˜0.1 wt % LAP for preparing ˜10 wt % PEGNB hydrogels. To vary macromer concentrations, and in some embodiments, ˜20 wt % PEGNB, ˜20 mM dithiol linker, ˜0.1 wt % LAP were mixed for preparing ˜20 wt % PEGNB hydrogels, and ˜30 wt % PEGNB, ˜30 mM dithiol linker, ˜0.1 wt % LAP were mixed for preparing ˜30 wt % PEGNB hydrogels.

In another example, a PEGDA hydrogel-forming solution is mixed to a final concentration of ˜10 wt % PEGDA (Mₙ≈3400 Da, JenKem Technology) and ˜0.1 wt % LAP. To vary PEGDA concentrations, ˜20 wt % and ˜30 wt % PEGDA can be mixed with ˜0.1 wt % LAP for polymerization.

In some embodiments, a hydrogel that encapsulates or disperses one or more cells has a diameter of about 0.5 μm to about 1,000 μm, such as from about 50 μm to about 500 μm, such as from about 100 μm to about 450 μm, such as from about 150 μm to about 400 μm, such as from about 200 μm to about 350 μm, such as from about 250 μm to about 300 μm. In some examples, a diameter of the hydrogel is from about 100 μm to about 180 μm. In at least one embodiment, the diameter (in μm) is about 1, about 5, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, or about 500, or ranges thereof. Any of the foregoing numbers can be used singly to describe an open-ended range or in combination to describe a close-ended range. Larger or smaller diameters are contemplated. The diameter of the hydrogels is determined as described in the Examples.

Compositions described herein can be characterized in terms of the amount or percent of dispersed or encapsulated cells released from the hydrogel after a certain amount of time. In some examples, the percent of cells released after about 48 hours can be about 1% or more, 99% or less, or combinations thereof, such as from about 1% to about 99%, such as from about 5% to about 80%, such as from about 10% to about 70%, such as from about 15% to about 50%, such as from about 20% to about 40%. In at least one embodiment, the percent of cells released after about 48 hours can be about 80% or less, such as about 60% or less, such as about 50% or less, such as about 40% or less, such as about 30% or less, such as about 25% or less, such as about 20% or less, such as about 15% or less, such as about 10% or less, such as about 5% or less. In some embodiments, the percent (%) of cells released after about 48 hours can be 1, 3, 5, 10, 15, 20, 25, 30, 35, 40, 45, 50, 55, 60, 65, 70, 75, 80, 85, 90, 95, or 99, or ranges thereof, though other amounts are contemplated. Each of the foregoing numbers can be preceded by the word “about,” “at least about,” “less than about,” or “more than about,” and any of the foregoing numbers can be used singly to describe an open-ended range or in combination to describe a close-ended range. Measurement of the amount and percent of cells released is determined as described in the Examples.

Compositions described herein can be characterized in terms of the amount or percent of cells remaining viable for a certain amount of time after dispersion or encapsulation within the hydrogel. In some examples, the percent of cells remaining viable for about 300 hours or more after dispersion or encapsulation within the hydrogel is about 5% or more, 100% or less, or combinations thereof, such as from about 5% to about 99%, such as from about 15% to about 95%, such as from about 30% to about 90%, such as from about 60% to about 85%. In at least one embodiment the percent of cells remaining viable for about 300 hours or more after dispersion or encapsulation within the hydrogel is about 80% or more, such as about 90% or more, such as about 95% or more. In some embodiments, the percent (%) of cells remaining viable for about 300 hours or more after dispersion or encapsulation within the hydrogel can be 5, 15, 20, 25, 30, 35, 40, 45, 50, 55, 60, 65, 70, 75, 80, 85, 90, 95, 99, or 100, or ranges thereof, though other amounts are contemplated. Each of the foregoing numbers can be preceded by the word “about,” “at least about,” “less than about,” or “more than about,” and any of the foregoing numbers can be used singly to describe an open-ended range or in combination to describe a close-ended range. Measurement of the amount and percent of cells percent of cells remaining viable for a certain amount of time after dispersion or encapsulation within the hydrogel is determined as described in the examples.

### Methods

Embodiments described herein also generally relate to methods of forming compositions described herein such as hydrogel-encapsulated/dispersed cells. The hydrogel-encapsulated/dispersed cells can be used as at least a portion of a composition, for example, for controlling production of secretomes. Additionally, or alternatively, the hydrogel-encapsulated/dispersed cells can be used as at least a portion of a controlled release composition. Other applications are contemplated and described herein.

In an embodiment, a method of forming a composition described herein includes introducing a hydrogel forming solution with one or more cells to form a mixture. The hydrogel forming solution and the cells can be introduced to a device (for example, a microfluidic device described herein or other device) in order to form the hydrogel. In some examples, the apparatus is a microfluidic device having a channel in which the mixture can flow. This channel can be a temporary channel. As described above, the hydrogel forming solution, can include one or more photoreactive monomers, one or more linkers, one or more photoinitiators, other components, and/or solvent(s). The hydrogel forming solution and cells can be suspended in an aqueous carrier is passed through a flow focusing, cross flow, or similar microfluidic channel to produce highly monodisperse, cell-laden microparticles.

The method further includes reacting the mixture to form the hydrogel. The reaction can take the form of “click” chemistry, polymerization, click polymerization, and/or curing such that components of the reaction mixture react. For example, the mixture can be polymerized by exposure to ultraviolet light, under polymerization conditions, to form the composition comprising the hydrogel-encapsulated/dispersed cell(s).

Here, after formation of the cell-laden microparticles (e.g., the hydrogel forming solution and the cell(s)), the microparticles are exposed to light (UV or visible) at a desired wavelength or wavelength range, such as a wavelength or wavelength range of about 290 nm to about 500 nm, such as from about 320 nm to about 460 nm, such as from about 340 nm to about 440 nm, such as from about 360 nm to about 420 nm, such as from about 380 nm to about 400 nm or from about 400 nm to about 420 nm, such as about 365 nm or about 405 nm, for varying timespans. In some embodiments, the wavelength or wavelength range of light is from about 290 nm to about 460 nm, such as from about 350 nm to about 450 nm, such as from about 375 nm to about 425 nm. The wavelength or wavelength range can be constant or varying during polymerization. Any of the foregoing numbers can be used singly to describe an open-ended range or in combination to describe a close-ended range. It is contemplated that other wavelengths of light can be used with appropriate reacting photoinitiators.

In some embodiments, polymerization conditions can include one or more of the following parameters:

A duration of exposure to ultraviolet light can be from about 1 millisecond to about 60 seconds, such as from about 5 milliseconds to about 50 seconds, such as from about 50 milliseconds to about 45 seconds, such as from about 100 milliseconds to about 40 seconds, such as from about 0.5 seconds to about 30 seconds, such as from about 1 second to about 20 seconds. Any of the foregoing numbers can be used singly to describe an open-ended range or in combination to describe a close-ended range. Other durations are contemplated.

An energy density of the ultraviolet light can be from about 1 mW/cm² to about 10,000 mW/cm², such as from about 10 mW/cm² to about 1,000 mW/cm², such as from about 50 mW/cm² to about 500 mW/cm², such as from about 75 mW/cm² to about 150 mW/cm², such as from about 80 mW/cm² to about 120 mW/cm². Any of the foregoing numbers can be used singly to describe an open-ended range or in combination to describe a close-ended range. Other energy densities are contemplated.

The pH of the reaction mixture during polymerization can be from about 5 to about 9, such as from about 6 to about 8, such as from about 6.5 to about 7.5. Any of the foregoing numbers can be used singly to describe an open-ended range or in combination to describe a close-ended range. Other pH values are contemplated.

Adjusting the initial cell titer, channel dimensions, and flow rate can be used to precisely control microparticle size and cell concentration in an independent manner. These microparticles can be created in sizes ranging from, for example, about 1 μm to about 2000 μm, such as from about 2 μm to about 1000 μm, such as from about 4 μm to about 500 μm, with cell concentrations ranging from, for example, 1 cell per microparticle to thousands of cells per microparticle, or more. The fabrication processes described can enable control of the physical, chemical, and mechanical characteristics of the apparatus's microenvironment by changing the size, shape, concentration, and number of reactive groups of the PEG-based and non-PEG-based monomer species. The processes additionally can enable control of these properties through data-informed adjustment of, e.g., the concentration of the components in the hydrogel forming solution and the UV light exposure duration and intensity. Adjusting these individual parameters, as well as others, can enable control of, e.g., the crosslinking density, pore size, and mechanical properties of the hydrogels.

FIG. 1C is a flowchart showing selected operations of an example of a method 170 for forming hydrogel-encapsulated/dispersed cells or compositions comprising hydrogel-encapsulated/dispersed cells according to at least one embodiment of the present disclosure.

Method 170 can be performed in a device such as the device 100 or the device 150. However, it is contemplated that any suitable device or tool can be used to form the hydrogel-encapsulated/dispersed cells or compositions comprising hydrogel-encapsulated/dispersed cells such as a droplet generator, emulsion, or other device.

Method 170 begins at operation 172 with introducing cells (e.g., one or more cells, such as MSCs) with one or more components in, e.g., the device 100 or the device 150, to form a reaction mixture. The cells can be in the form of a suspension in an aqueous buffer such as phosphate buffered saline (PBS). The one or more components can include one or more photoreactive monomers, one or more linkers (e.g., dithiol linker), one or more photoinitiators, and/or one or more solvents. Other materials such as reagents, catalysts, and/or cell-adhesion peptides can be optionally added. The reaction mixture can be in the form of microparticles in solution in the presence of the oil. These microparticles can be created in sizes ranging from, e.g., about 1 μm to about 2000 μm, such as from about 2 μm to about 1000 μm, such as from about 4 μm to about 500 μm, with cell concentrations ranging from, e.g., 1 cell per microparticle to thousands of cells per microparticle, or more. Other sizes of microparticles and concentrations of cells are contemplated.

Operation 172 can include flowing a hydrogel forming solution into the device 100, 150 at a flow rate of about 0.1 μL/min to about 150 μL/min, such as from about 25 μL/min to about 125 μL/min, such as from about 50 μL/min to about 100 μL/min, such as from about 80 μL/min to about 100 μL/min. In some examples, the flow rate of the hydrogel forming solution into the device can be about 0.1 to about 15 μL/min, such as from about 0.5 μL/min to about 10 μL/min, such as from about 1 μL/min to about 5 μL/min. Any of the foregoing numbers can be used singly to describe an open-ended range or in combination to describe a close-ended range. Higher or lower flow rates are contemplated

In some embodiments, and when the hydrogel forming solution does not include cells, operation 172 can further include flowing a cell stream—one or more cells in a suspension or suitable media such as a buffer, such as PBS—into the device 100, 150 at a flow rate of about 0.1 μL/min to about 150 μL/min, such as from about 25 μL/min to about 125 μL/min, such as from about 50 μL/min to about 100 μL/min, such as from about 80 μL/min to about 100 μL/min. In some examples, the flow rate of the hydrogel forming solution into the device can be about 0.1 to about 15 μL/min, such as from about 0.5 μL/min to about 10 μL/min, such as from about 1 μL/min to about 5 μL/min. Any of the foregoing numbers can be used singly to describe an open-ended range or in combination to describe a close-ended range. Higher or lower flow rates are contemplated for this cell stream. In some embodiments, the hydrogel forming solution and the cell-stream are introduced at the same time or separate times to the same or different openings of the device 100, 150.

At operation 174, an oil (e.g., a fluorocarbon oil) can be introduced to the reaction mixture. Upon introduction, the oil with the reaction mixture can form droplets. Here, for example, the oil is added to the device 100, 150, and the oil can aid in the formation of droplets within the fluidic channel. Such droplets can help, e.g., bring together the polymerizable reactants and the cells. A flow rate of the oil into the device 100, 150 can be from about 0.1 μL/min to about 200 μL/min, such as from about 1 μL/min to about 150 μL/min, such as from about 25 μL/min to about 125 μL/min, such as from about 50 μL/min to about 100 μL/min, such as from about 80 μL/min to about 100 μL/min. In some examples, the flow rate of the cell stream into the device can be about 0.1 to about 15 μL/min, such as from about 0.5 μL/min to about 10 μL/min, such as from about 1 μL/min to about 5 μL/min. Any of the foregoing numbers can be used singly to describe an open-ended range or in combination to describe a close-ended range. Higher or lower flow rates are contemplated for this oil stream.

The method 170 further includes polymerizing the reaction mixture to form a hydrogel-encapsulated/dispersed cell, or compositions thereof, at operation 176. The polymerization reaction of operation 176 can be performed under polymerization conditions such as those described herein. Polymerization of the reaction mixture forms the hydrogel-encapsulated/dispersed cells, compositions comprising hydrogel-encapsulated/dispersed cells, or combinations thereof. In some embodiments, the pH of the reaction mixture before, during, and/or after polymerization can be from about 5 to about 9, such as from about 6 to about 8, such as from about 6.5 to about 7.5. Any of the foregoing numbers can be used singly to describe an open-ended range or in combination to describe a close-ended range. Other pH values are contemplated.

Polymerization conditions can include exposing the reaction mixture to UV light at a desired wavelength or wavelength range, such as a wavelength or wavelength range of about 290 nm to about 500 nm, such as from about 320 nm to about 460 nm, such as from about 340 nm to about 440 nm, such as from about 360 nm to about 420 nm, such as from about 380 nm to about 400 nm or from about 400 nm to about 420 nm, such as about 365 nm or about 405 nm, for varying timespans. In some embodiments, the wavelength or wavelength range of UV light is about 350 nm to about 450 nm, such as from about 375 nm to about 425 nm. The wavelength or wavelength range can be constant or varying during operation 176. Any of the foregoing numbers can be used singly to describe an open-ended range or in combination to describe a close-ended range. Other wavelengths or wavelength ranges are contemplated. The source of the UV light can be the polymerization control device 105 or the polymerization control device 155. It is contemplated that other wavelengths of light can be used with appropriate reacting photoinitiators.

The polymerization conditions of operation 176 can further include a duration of exposure to the UV light. Such durations can be 1 millisecond (ms) or more and/or about 5 min. or less, such as from about 1 ms to about 60 seconds (s), such as from about 5 ms to about 50 seconds, such as from about 50 ms to about 45 seconds, such as from about 100 ms to about 40 seconds, such as from about 0.5 seconds to about 30 seconds, such as from about 1 second to about 20 seconds. Any of the foregoing numbers can be used singly to describe an open-ended range or in combination to describe a close-ended range. Shorter or longer durations of exposure to UV light are contemplated.

An energy density of the UV light for the polymerization conditions of operation 176 can be from about 1 mW/cm² to about 10,000 mW/cm², such as from about 10 mW/cm² to about 1,000 mW/cm², such as from about 50 mW/cm² to about 500 mW/cm², such as from about 75 mW/cm² to about 150 mW/cm², such as from about 80 mW/cm² to about 120 mW/cm². Any of the foregoing numbers can be used singly to describe an open-ended range or in combination to describe a close-ended range. Higher or lower energy densities are contemplated. The energy density can be constant or varying during operation 176.

The polymerization process described herein can improve cell viability over conventional techniques. For example, upon photoinitiation, a homogenous hydrogel network with reduced network contraction relative to other equivalent materials reduces stress imparted on encapsulated/dispersed cells. In addition, it is believed that the polymerization described herein can mitigate ROS through active participation in the cross-linking mechanism of, e.g., PEGNB, contributing to the polymerization of the network rather than removing electrons from cellular membranes and destabilizing them, which can kill or contribute to cell death. In polymerizations with PEGDA, ROS can be mitigated by purging oxygen from the microenvironment via a non-reactive or inert gas which is free or substantially free of oxygen can be used, such as nitrogen and noble gases (e.g., argon). For polymerizations using mixtures of PEGDA and PEGNB, ROS can be mitigated by the addition of PEGNB and its above properties, but can be further mitigated if necessary through purging of the microenvironment with inert gas.

Release of the one or more cells from the hydrogel can be controlled by embodiments described herein.

In some cases, the combination of PEGNB with another photoreactive monomer, such as PEGDA, enables physical and chemical tuning of the droplet environment to optimize cell viability and excretion of, e.g., secretomes such as cytokines. The encapsulation/dispersion process and resultant hydrogel can maintain cell viability longer than non-encapsulated/dispersed counterparts, and can localize cells at a target location by temporarily preventing their migration.

After polymerization, the hydrogel-encapsulated/dispersed cells and/or compositions comprising the hydrogel-encapsulated/dispersed cells can be purified or otherwise isolated from the other materials exiting the device 100, 150.

In some embodiments, the one or more cells, dispersed in or encapsulated within a hydrogel have improved viability or lifetime relative to conventional methods. For example, the cells dispersed in or encapsulated within a hydrogel as described herein have cell viability of about 1 hour or more after encapsulation/dispersion, such as about 5 hours or more, such as about 10 hours or more, such as about 24 hours or more, such as about 36 hours or more, such as about 48 hours or more, such as about 60 hours or more, such as about 72 hours or more, such as about 84 hours or more, such as about 96 hours or more, such as about 108 hours or more, such as about 120 hours or more, such as about 132 hours or more, such as about 144 hours or more, such as about 156 hours or more, such as about 168 hours or more, such as about 180 hours or more, such as about 192 hours or more, 204 hours or more, such as about 216 hours or more, such as about 228 hours or more, such as about 240 hours or more after encapsulation/dispersion of the cells in the hydrogel. Shorter or longer time periods are contemplated.

The methods described herein can provide a high-throughput route to transplantable cells for the treatment of diseases, and the resulting hydrogel-encapsulated/dispersed cells show, e.g., an enhanced ability to control secretomes such as cytokines. The methods can also provide controlled release compositions by tailoring, for example, the components of the hydrogel.

Embodiments described herein can also enable control over hydrogel size and shape, via the manipulation of, e.g., relative flow velocity of immiscible phases, nozzle geometry, and nozzle dimension. The methods described herein promote long-term cell viability after encapsulation/dispersion as well as the protection of encapsulated/dispersed cell from external deleterious factors such as innate immune responses and shear stress. The microparticle length scale also enables enhanced exchange of nutrients, waste, and secreted biomolecules to and from the cells and its surrounding environment, in contrast to other conventional encapsulation/dispersion methods.

In some embodiments, processes for forming the hydrogel-encapsulated cells involve the suspension of therapeutic cells including mesenchymal stem cells, bone marrow aspirate, etc. in, for example, a photoreactive monomer and dithiol linker solution of varying concentrations, molecular weights, and molecular geometries. In at least one embodiment, PEGDA, such as 20 k 4-arm PEGNB, is used as a monomer. As described above, PEGNB polymerizes via a step-growth reaction occurring between its “ene” group and a thiol group (of a thiol containing monomer). The polymerization mitigates reactive oxygen species. Upon photoinitiation, a homogenous hydrogel network with reduced network contraction to other equivalent materials and thus further reduces stress upon encapsulated cells. It is believed that ROS is also mitigated though active participation in the crosslinking mechanism of PEGNB, contributing to the polymerization of the network rather than removing electrons from cellular membranes and destabilizing them, which is what kills or contributes to cell death. In PEGDA, ROS is mitigated by purging oxygen from the microenvironment via a non-reactive or inert gas which is free or substantially free of oxygen can be used, such as nitrogen and noble gases (for example, argon). In mixtures of PEGDA and PEGNB, ROS is mitigated by the addition of PEGNB and its above properties, but can be further mitigated if necessary through purging of the microenvironment with inert gas.

In some cases, the combination of PEGNB and PEGDA enable physical and chemical tuning of the droplet environment to optimize cell viability and excretion of secretomes such as cytokines. The encapsulation process maintains cell viability longer than non-encapsulated counterparts, and localizes cells at a target location by temporarily preventing their migration.

Each of U.S. patent application Ser. No. 17/463,362, filed Aug. 31, 2021 (now published as U.S. Patent Application Publication No. 2022/0064624), U.S. patent application Ser. No. 17/482,143, filed Sep. 22, 2021 (now published as U.S. Patent Application Publication No. 2022/0089821), and U.S. patent application Ser. No. 17/484,381, filed Sep. 24, 2021 (now published as U.S. Patent Application Publication No. 2022/0090000), are incorporated herein by reference in their entireties.

The following examples are put forth so as to provide those of ordinary skill in the art with a complete disclosure and description of how to make and use embodiments of the present disclosure, and are not intended to limit the scope of embodiments of the present disclosure. Efforts have been made to ensure accuracy with respect to numbers used but some experimental errors and deviations should be accounted for.

Further, while the present disclosure refers to “microcapsules” and “microgels” it will be appreciated that the disclosure may be applied to capsules and gels having a smaller size (for example, “nanocapsules” or “nanogels”) or capsules and gels having a larger size (for example, “macrocapsules” or “macrogels”). In addition, while the examples are described in terms of cells, it is contemplated that other objects, including biomolecules, can be utilized. For example, the methods described herein can be used to form hydrogel-encapsulated biomolecules and used to form therapeutic doses of hydrogel-encapsulated biomolecules. Also, while examples and embodiments are described herein with reference to hydrogel encapsulation of cells, it is contemplated that the cells can be dispersed, retained, or otherwise held in the hydrogels. For example, device 100 and device 150 can be utilized to form hydrogels having cells dispersed therein, and processes for forming the hydrogel-encapsulated cells can be used to form hydrogels having cells dispersed therein.

### Examples

### Device for Forming Hydrogel-Encapsulated/Dispersed Cells

Microfluidic devices, such as those illustrated in FIGS. 1A and 1B, were fabricated. Microfluidic flow networks were fabricated using standard soft lithography techniques. Briefly, about 10 g polydimethylsiloxane (PDMS, Sylgard 184, Dow Corning USA) was poured onto SU-8 photoresist patterned silicon master wafers for droplet generation and polymerization devices, vacuumed for about 30 minutes to remove the entrapped air, and then transferred to an oven (temperature of about 70° C.) to cure for about 8 to 24 hours. The PDMS replicas were then trimmed and punched with a sharpened 20G dispensing needle (CIVIL Supply, USA) to fashion inlets and outlets. Alternatively, the inlets and outlets can be pre-formed into the PDMS by attaching/inserting posts of the desirable inlet/outlet size into the molds where inlet/outlets are placed.

The PDMS replicas were cleaned with ethanol, and then exposed to oxygen plasma (Harrick Scientific, USA), placed in conformal contact with clean glass slides, and transferred to an oven (temperature of about 70° C.), and remained in the oven for about 8 to 24 hours to form an irreversible bond between the PDMS microfluidic replicas and the glass slides or glass coverslips. The droplet generation device was connected with the polymerization device with tygon tubing (United States Plastic Corporation, USA).

For the droplet generation device of FIG. 1A, channel dimensions (height×width, h×w) were 50 μm×80 μm for the aqueous stream, and 50 μm×30 μm for the oil stream. Dimensions (h×w) for the polymerization device of FIG. 1A were 120 μm×120 μm. For the polymerization scheme utilizing crosslinker tuning shown in FIG. 1B, dimensions (h×w) were 80 μm×80 μm for the aqueous stream, and 80 μm×20 μm for the oil stream.

### Linker Length

Linker length was investigated. Hydrogels formed via free radical-mediated thiol-ene step-growth photopolymerization have been developed for a broad range of tissue engineering and regenerative medicine applications. While the crosslinking mechanism of thiol-ene hydrogels has been well-described, there has been only limited work exploring the physical differences among gels arising from variations in crosslinker properties. Conventional efforts to fabricate hydrogels for in situ cell encapsulation typically involve Michael-type addition reactions between thiol and alkene groups, and control over gelation kinetics via stepwise polymerization. Application of this polymerization scheme, while versatile and biocompatible, is ultimately constrained by its relatively slow hydrogel formation kinetics. This limitation presents various challenges to the fabrication and application of hydrogels such as miniaturizing hydrogel features, achieving spatial and temporal control over gelation, and the inability to rapidly fabricate microscale features.

Further, limited effort has been made to explore the effect of linear linker length on thiol-ene hydrogel formation and crosslinking efficiency, as well as on formed hydrogel properties. As such, the relationship between the macromolecular crosslinking reaction and hydrogel formation during photopolymerization has compromised precise control over hydrogel properties and constrained potential applications. For example, the on-chip polymerization of PEGNB particles has been frustrated by the material's slow gelation rate and the short times available for photopolymerization, which arise from the large droplet velocities and small available exposure areas. These material limitations have led to the use of lower throughput batch or quasi-batch microgel fabrication processes to increase exposure time. Accordingly, high-volume manufacturing has been a challenge due to, for example, consistency and stability of the formed hydrogels.

Inconsistent microcapsule properties or the unpredictable functional response or condition of encapsulated cells to uncontrolled polymerization tremendously limits the applicability of PEGNB (or other macromers) as a cell carrier. Microparticle polymerization in elongated channels has been attempted to address this particular concern, but introduces instabilities that lead to variable droplet transit times and therefore exposure times, which could lead to low reproducibility and efficiency. Additionally, sufficient microparticle gelation heavily depends on the exposure time, which is governed by, for example, the absolute velocity of droplets and microchannel geometry and dimension. Because these parameters cannot be decoupled, the tunability of on-chip polymerization is ultimately determined by, for example, the flow rates utilized for drop formation. These process limitations, which have largely prevented the high-throughput miniaturization of PEGNB gels, likely arise from PEGNB's slow gelation kinetics.

There is a need for a greater understanding of thiol-ene chemistry and its relationship to gelation rates to achieve rapid and continuous fabrication of microcapsules on-chip. For example, while reports describe the formation of PEGNB hydrogels using different length crosslinkers, the effects of linker length on gel formation rates and hydrogel properties have not been specifically addressed.

In some embodiments, PEGNB hydrogel microparticles (also referred to as microgels) are fabricated on-chip with great tunability in particle size and shape. In some examples, changing the PEG-dithiol linker length significantly affects hydrogel formation rates by tuning the crosslinking efficiency. Additionally, the formed hydrogel properties are affected by linker length. While not wishing to be bound by theory, it is believed that these differences result from the progression of the hydrogel network architecture as the PEGNB or other photoreactive monomer becomes crosslinked by dithiol linkers of different lengths. In some examples, shorter PEG-dithiol linkers have a higher likelihood of reacting with -enes from the same PEGNB molecule, presumably due to their inability to reach a neighboring PEGNB molecule. This state leads to ‘linker neutralization’ and limited-to-no cross-linking. Additionally, and in some examples, disulfide formation and ‘self-termination’ of the linker occurs at higher rates for shorter linkers. Linker self-termination reduces crosslinking and produces a stoichiometric mismatch between thiols and -enes in the hydrogel-forming solution.

In addition, the formed hydrogels are cytocompatible. In some examples, greater than about 95% cell viability was initially achieved under all dispersion/encapsulation scenarios, indicating that droplet formation does not alter cell viability. In addition, about 85% of encapsulated cells survived after 14 days of culture, illustrating that the microfluidic platforms and hydrogel-forming schemes described herein are effective for cell encapsulation/dispersion.

In some embodiments, the type and size of linker used to crosslink photoreactive monomers (for example, multi-arm PEGNB) is used to tune hydrogel formation kinetics and the equilibrium hydrogel network architecture. While not wishing to be bound by theory, it is hypothesized that under similar reaction conditions, a more accessible linker improves network ideality relative to shorter linker. Accordingly, the longer linkers can consequently promote significantly more rapid macromer crosslinking and gelation.

Accelerated gel formation satisfies an urgent unmet need for rapid polymerization in droplet microfluidics. Embodiments herein demonstrate the ability to photopolymerize PEGNB and other photoreactive monomers under flow on a microfluidic chip, with reliable control over the resulting microgel size and shape in a high-throughput manner. Further, encapsulation/dispersion of cells, and subsequent culture of the hydrogel-encapsulated/dispersed cells shows excellent viability over long durations, demonstrating that that hydrogel dynamics can be readily customized to fulfill a variety of needs in, for example, tissue engineering, controlled cell delivery, or drug release applications.

### Photoinitiator and Macromer Synthesis

The initiator species, lithium acylphosphinate (LAP), was synthesized according to the following procedure. About 3.0 g of 2,4,6-trimethylbenzoyl chloride (Sigma Aldrich, USA) was added dropwise to a 250 mL round-bottom flask containing an equimolar amount of dimethyl phenylphosphonite and stirred at about room temperature under nitrogen for about 8 to 24 hours. A four-fold (mol:mol) excess of lithium bromide (LiBr, Sigma Aldrich, USA) dissolved in about 100 mL of methyl ethyl ketone (MEK, Sigma Aldrich, USA) was added to the round-bottom flask and the resulting mixture was heated to about 50° C. for about 10 minutes. White crystalline salts were formed upon cooling to about room temperature over a period of about 8 to 24 hours. Product crystals were filtered on a Buchner funnel and rinsed with ice-cold MEK, then placed under vacuum until a constant weight was achieved. LAP was confirmed via 400 MHz proton nuclear magnetic resonance (NMR) using a suitable deuterated solvent, such as deuterated toluene (toluene-d8).

The hydrogel-forming macromer, PEGNB, was synthesized according to established procedures or purchased. Briefly, about 12 g of 4-arm PEG (MW, 20,000 Da, Creative PEGworks, USA) and about 20 mL of MeCl₂ was placed in a 40 mL scintillation flask and dissolved at about room temperature (15° C.-25° C.) while stirring. Into a separate 50 mL round-bottom flask was added a desired amount of N,N-dicyclohexylcarbodiimide (DCC, Sigma Aldrich, USA) followed by dissolution in about 10 mL of MeCl₂. To this solution was added a desired amount of 5-norbornen-2-carboxylic acid (5N2B, Sigma Aldrich, USA). The previously prepared 4-arm PEG solution was added to the 50 mL round-bottom flask, placed in an ice bath, and stirred or otherwise mixed for a period of about 8 to 24 hours under nitrogen or argon purging. Ice-cold diethyl ether (Sigma Aldrich, USA) was added to the product-containing solution and the resultant precipitate was vacuum-filtered, recrystallized, and then subjected to soxhlet extraction for about 36 hours to remove undesired impurities. The PEGNB product was dried for about 8 to 24 hours under vacuum. The product was recovered with about 77% yield, and confirmed via 400 MHz proton NMR using d2-DMSO as solvent.

Determination of Serpentine Channel Loops for Gelation of Microparticles with the 1.5 kDa PEG-Dithiol Linker

Achieving on-chip polymerization of PEGNB with a 1.5 kDa PEG-dithiol linker has been challenging due to, for example, the UV exposure area through which the droplets pass through, the mismatch of polymerization kinetics, and the exposure time for polymerization. A batch polymerization, where the reaction occurs in a microcentrifuge tube with the use of an external UV source has been utilized to increase exposure time as a compensation for slow reaction kinetics. However, a vertical UV intensity gradient is formed, which could result in non-uniform UV intensity and affect the cellular response from individual microparticles.

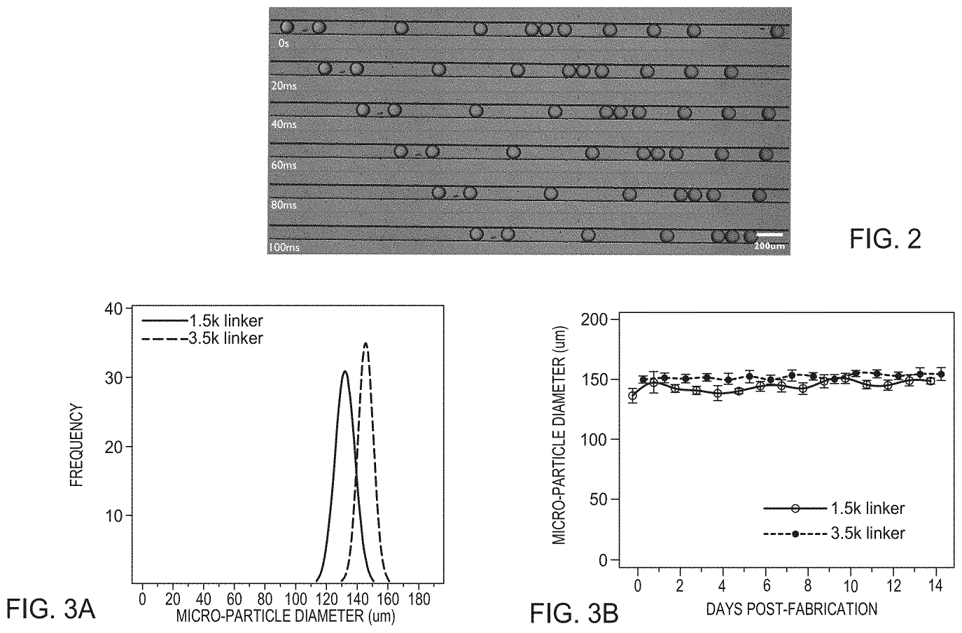

To achieve a standardized polymerization for enabling uniform physical properties of microparticles, the number of loops for sufficient gelation was determined by the following non-limiting procedure. When the flow rates for the macromer stream (the hydrogel-forming solution), the PBS stream (the cell stream), and the oil stream were fixed at about 0.5 μL/min, about 0.5 μL/min, and about 5 μL/min, respectively, droplets traveling along the polymerization device were video recorded. Images of the droplets traveling along the polymerization device are shown in FIG. 2. The images were extracted at times of about 0 ms, about 20 ms, about 40 ms, about 60 ms, about 80 ms, and about 100 ms.

By tracking ˜100 droplets traveling within a fixed time frame of, for example, about 100 milliseconds (ms), absolute velocity was calculated to be about 12 μm/ms by dividing the distance traveled by 100 ms. Absolute velocity multiplied by the UV exposure time (which is fixed at about 20 seconds), approximates the total distance for sufficient gelation of which is about 240 millimeters. Because the serpentine channel length has a fixed value of about 20 millimeters, the number of loops for sufficient gelation with a particular design can be determined. As an example, the number of loops for gelation of microparticles with the 1.5 kDa PEG-dithiol linker is about 15 loops.

It is contemplated that more or less loops can be used for various linkers depending on, for example, the size of the linker and the photoreactive monomer, among other parameters.

### Fabrication and Recovery of Microparticles

Prior to droplet generation and polymerization, the microfluidic devices were treated with Aquapel™ (PPG Industries, USA) to facilitate droplet generation via the establishment of a hydrophobic surface. For the polymerization device scheme of FIG. 1A, a hydrogel forming solution that includes about 20 wt % PEGNB (MW 20,000 g/mol), about 20 mM PEG-dithiol linker (MW 1500 kDa, Sigma Aldrich, USA), and about 0.2 wt % LAP were mixed in a vortex mixer for about 60 seconds to ensure sufficient mixing was utilized. The hydrogel forming solution and the phosphate buffered saline (PBS) were then injected into the microfluidic channels at approximately the same rate, for example, if the hydrogel forming solution is injected at ˜1 μl/min then the PBS solution is injected at ˜1 μl/min. The two solutions flow into the microfluidic device and are mixed after the solutions enter the chip, resulting in the dilution of the hydrogel forming solution to a final concentration of about 10 wt % PEGNB, about 10 mM PEG-dithiol linker, and about 0.1 wt % LAP.

Flow rates for the aqueous phase and the oil phase were held constant at about 1 μL/min and about 5 μL/min in droplet generation device. A UV lamp (for example, OmniCure™ lamp) was set up with 100% intensity and a fixed height to generate a final UV intensity of about 100 mW/cm² for polymerizing particles on the polymerization device of FIG. 1A.

Of note, the height of the UV lamp from the device can be adjusted. Suitable height ranges can be from about 0.5 in to about 24 in for the lamp used in this experiment. It is contemplated that the distance can be adjusted to obtain the desired crosslinking by changing the intensity of the light source that is interacting with the hydrogel. A source with variable intensity could also be used.

For the linker length-mediated polymerization tuning device of FIG. 1B, about 20 wt % PEGNB (MW 20,000), about 20 mM PEG-dithiol linker (MW 3500, Jenkem, China), and about 0.6 wt % LAP were sufficiently mixed and injected into microfluidic channels together with PBS. The flow rate for the aqueous phase and the oil phase were held constant at about 1 μL/min and about 10 μL/min, respectively. A 40× objective lens with a DAPI (4,6-diamidino-2-phenylindole) filter cube was used for the photopolymerization. About 2 wt % of surfactant (for example, Pico Surf™) was mixed in the fluorocarbon oil to facilitate droplet pinch-off. Particles were then separated from the oil via centrifugation on a 10 μm cell strainer (PluriSelect™, Germany), and then transferred to PBS containing about 0.1 wt % of a surfactant (for example, Pluronic™ F-68, Sigma Aldrich, USA).

### Determination of Droplet Velocity and UV Exposure Time on the Device

Under a constant flow rate on the droplet generation device for generating stable, monodisperse droplets, the absolute velocity of droplets traveling on the polymerization device was monitored via a high-speed camera (Phantom™ V310) and calculated. For this experiment, a device of FIG. 1A was utilized. Total distance enabling sufficient polymerization was determined by multiplying absolute velocity by pre-determined exposure time of about 20 seconds. With a length of the serpentine channel fixed as 20 millimeters, the number of loops were calculated by dividing the total distance by the channel length. As an example, the number of loops for polymerization of the PEGNB and 1.5 kDa PEG-dithiol linker is about 12 loops.

As an example, and using a device of FIG. 1B, no loops are utilized for the polymerization of the PEGNB and 3.5 kDa PEG-dithiol linker. It is contemplated that loops can be utilized if desired.

### Microparticle Characterization

The polymerized MSC-laden microparticles fabricated from both approaches were incubated in PBS at a temperature of about 37° C. after their collection from the chip. Images were taken every day for 14 days. The diameter of the polymerized MSC-laden microparticles was determined by measuring 100 microparticles with ImageJ (National Institutes of Health).

### MSC Culture and Encapsulation/Dispersion

Equine MSCs were isolated and cultured from bone marrow from a young adult horse as previously described. Primary MSCs were cultured in Dulbecco's modified Eagle's medium (DMEM; Life Technology, USA) with low glucose, and supplemented with ˜10 vol % fetal bovine serum (FBS, Life Technology, USA), ˜1 vol % antibiotic-antimycotic (Life Technology, USA), and ˜2 ng/ml FGF-2 (Sigma Aldrich, USA). Cells were cultured in an incubator at a temperature of about 37° C. under an atmosphere of about 5% CO₂, and the media was changed every ˜3 days and cell populations were sub-cultured when cell confluency reached about 70-80%. Prior to encapsulation/dispersion of the cells, the MSCs were trypsinized, pelleted, and resuspended to a cell density of about 1×10⁷ cells/mL. The density of the cell-carrying medium was adjusted to about 1.06 g/mL by adding in about 16% v/v OptiPrep™ (Iodixanol, Sigma Aldrich, USA). A general non-limiting procedure for trypsinizing, pelleting, and resuspending is as follows: Cells were removed from liquid nitrogen and quickly thawed in a 37° C. water bath then resuspended in about 10 mL DMEM-low glucose cell media, pelleted via centrifugation in a swinging bucket rotor at 400 g for ˜10 min at about 37° C., the supernatant removed, then resuspended in 10 mL DMEM-low glucose cell media supplemented with 10% FBS, 1% penicillin-streptomycin, ˜0.1% amphotericin B and ˜2 ng/mL FGF-2 under a sterile laminar hood/cabinet. The entire cell suspension was transferred to a T75 cell culture flask, and placed into an incubator at ˜37° C. and ˜5% CO_{2.}

To polymerize the PEGNB droplets within the polymerization device of FIG. 1A, the flow rates for the cell stream, the hydrogel-forming solution stream, and the oil stream were held constant at about 0.5 μL/min, about 0.5 μL/min, and about 5 μL/min, respectively.

To polymerize the PEGNB droplets via linker length-mediated polymerization tuning of FIG. 1B, the flow rates for the cell stream, the hydrogel-forming solution stream, and the oil stream were held constant at about 1 μL/min, about 1 μL/min, and about 10 μL/min, respectively. Thiolated Rhodamine B (0.01 wt %) was copolymerized together with PEGNB and the individual thiol crosslinker to label the hydrogel network. Recovery of the MSC-laden microparticles was conducted as described above. The recovered MSC-laden microparticles were then transferred and cultured within an ultra-low attachment 6-well plate (VWR, USA) in an incubator (˜37° C. and ˜5% CO₂).

To fabricate macroscale cell-laden hydrogels using the device of FIG. 1A, MSCs with a density of ˜1×10⁷ cells/mL were mixed with PEGNB, 1.5 kDa PEG-dithiol linker, and LAP to a final volume of ˜100 with a concentration of about 10 wt % PEGNB, about 10 mM PEG-dithiol linker, and about 0.1 wt % LAP, within a 1 mL syringe with the tip trimmed off After UV exposure (˜100 mW/cm²) for 20 seconds, cell-laden hydrogels were removed and cultured within an ultra-low attachment 6-well plate (VWR, USA) in the incubator (˜37° C. and ˜5% CO₂).

### MSC Viability Assay

Viability of the encapsulated/dispersed cells was measured by staining using a live/dead viability kit (Life Technologies, USA), which is a cellular membrane integrity assay that stains live cells with green fluorescence and dead cells with red fluorescence. Cell viability was imaged with an inverted fluorescence microscope (IX-71, Olympus, USA) and cell viability was calculated based on 100 cell-laden microparticles using ImageJ.

### MSC Release Measurement

The MSC-laden PEGNB microparticles generated with the above two approaches were collected separately for about 30 minutes onto two 40 μm cell strainers (Life Technologies, USA), which were then submerged into an ultra-low attachment 6-well plate (VWR, USA) containing ˜8 mL culture media (about 10 mL DMEM-low glucose cell media supplemented with ˜10% FBS, ˜1% penicillin-streptomycin, ˜0.1% amphotericin B, and ˜2 ng/mL FGF-2). Released cells were counted with a hemocytometer (Life Technologies, USA) after transferring cell strainers into new wells every day. To determine released cell viability and functionality, the released MSCs were either stained for measuring cell viability or transferred into a 6-well plate for culture.

### Gene Expression Analysis

Total RNA of monolayer equine MSCs, MSCs recovered from suspension culture, and macro- or micro-encapsulated MSCs from the same passage were extracted on day 1 and day 14. Monolayer MSCs, and MSC-laden microgels were homogenized manually in a homogenization tube and lysed in TRIzol™. RNA extraction and purification were performed with PureLink™ RNA Mini Kit (Life Technologies, USA) according to the manufacturer's protocol.

Reverse Transcription polymerase chain reaction (PCR) was performed to synthesize complementary deoxyribonucleic acid (cDNA) with Moloney Murine Leukemia Virus (M-MLV) reverse transcriptase (Promega, USA) according to manufacturer's protocol. Quantitative real-time polymerase chain reaction (qRT-PCR) was performed with iTaq™ Universal SYBR™ Green Supermix (Bio-Rad, USA) and an Applied Biosystems 7900 Real-Time PCR detection system (Bio-Rad, USA) per manufacturer's protocol to measure the relative expression of target genes, which were normalized to the expression of a housekeeping gene (constitutive gene for the maintenance of basic cellular function), β-actin. A comparative cyclic threshold (CT) method was used to determine relative expression levels of target genes under experimental conditions as to control conditions. The sequences of PCR primers were as follows. β-actin: 5′-ATGGATGATGATATCGCCG-3′ and 5′-CACGTATGAGTCCTTCTGG-3′; FGF-2: 5′-ACTTCAAGCAGAAGAGAGAG-3′ and 5′-CCGTAACACATTTAGAAGCC-3′; TGF-β: 5′-CTATAAGACTGTGGAGACCG-3′ and 5′-CTGTATTTCTGGTACAGCTC-3′; VEGF-α: 5′-CAGATTATGCGGATCAAACC-3′ and 5′-TTTGCAGGAACATTTACACG-3′. The relative expression levels of target genes were compared against monolayer MSCs on day 1 and day 14.

For the comparative or ΔΔCt method of quantitative polymerase chain reaction (qPCR) data analysis, the cycle threshold (the cycle number when the fluorescence of a PCR product can be detected above the background signal) values obtained from two different experimental RNA samples are directly normalized to a housekeeping gene and then compared. Then, the difference in the ΔCt values between the experimental and control samples ΔΔCt is calculated.

### Statistical Analysis

All statistical analyses were based on at least three experimental replicates. Statistical analysis was performed on gene expression data using student's t test (Graph Pad Prism). A P value of 0.05 was considered as the threshold for statistical significance.

### Characterization of Microparticles Fabricated On-Chip

FIGS. 3A and 3B show exemplary characteristics of the fabricated PEGNB microparticles using the polymerization scheme of FIG. 1A for the 1.5 kDa PEG-dithiol linker and the linker length-mediated crosslinking tuning scheme of FIG. 1B for the 3.5 kDa PEG-dithiol linker. Specifically, FIG. 3A shows that a narrow size distribution of polymerized PEGNB microparticles was recovered into an aqueous buffer following photopolymerization by each fabrication scheme. Because PEGNB is susceptible to hydrolysis due to the presence of ester bonds, degradation, as characterized by the equilibrium swelling ratio of microparticles, was measured by monitoring change in micro-particle diameter over 14 days. Results for the microparticle swelling over time are shown in FIG. 3B and were determined using ImageJ. The microparticles fabricated with the 1.5 kDa PEG-dithiol linker within the polymerization chamber (device of FIG. 1A) swelled by about 11% after 14 days, which was significantly higher than those fabricated with the 3.5 kDa PEG-dithiol linker via the linker length-mediated crosslinking tuning (device of FIG. 1B), whose diameter only increased by about 2%.

These observations show that instead of decreasing mesh size by using shorter linkers, the 1.5 kDa PEG-dithiol linker can result in a looser mesh due to gelation defects. The ester bonds of the shorter linker hydrolyze quicker, causing more swelling and more cells released. This may be due to, for example, the lower number of ester bonds in the shorter linker, a lower degree of entanglement when using the shorter linker, and the higher amount of auto-termination when using the shorter linker.

### Cell Microencapsulation and Post-Encapsulation Cell Viability

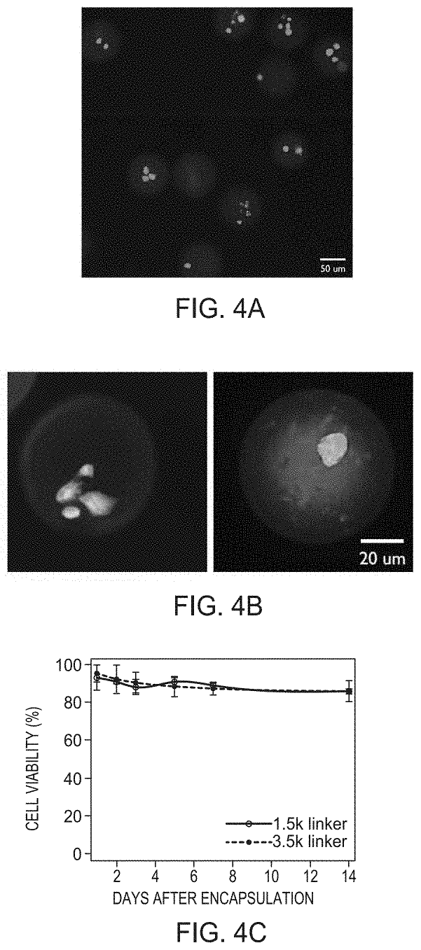

To investigate the effect of the two fabrication schemes on cytocompatibility, MSC-laden microparticles were cultured after recovery into aqueous phase and monitored for cell viability over 14 days. FIGS. 4A and 4B show exemplary images of encapsulated MSCs using a 4× objective lens and a 20× objective lens, respectively. FIG. 4C is exemplary data for the example hydrogel-encapsulated/dispersed cells made from a 1.5 kDa PEG-dithiol linker or a 3.5 kDa PEG-dithiol linker. Excellent initial cell viability was achieved with about 95% of the cells maintaining viable post-encapsulation for both fabrication schemes. Cell viability only decreased slightly to about 90% after 14 days of culture, indicating that the on-chip mixing and polymerization had minimal toxic effects on the cells. The results also demonstrated that PEGNB microparticles as formed by methods described herein excel at supporting cells with a suitable physiological environment.

### Release of Encapsulated MSCs

The use of the 3.5 kDa PEG-dithiol linker can improve gelation efficiency better than the 1.5 kDa PEG-dithiol linker, resulting in stiffer hydrogels and diminished mesh size when each arm of PEGNB molecule has a molecular weight of about 5,000 Da. The effects of such hydrogel characteristics on cell-release profiles from the perspective of potential cell delivery-based therapies was investigated as precise control of cell release from the hydrogel scaffold could significantly improve clinical treatment by timely matching with pharmaceutical scheduling.

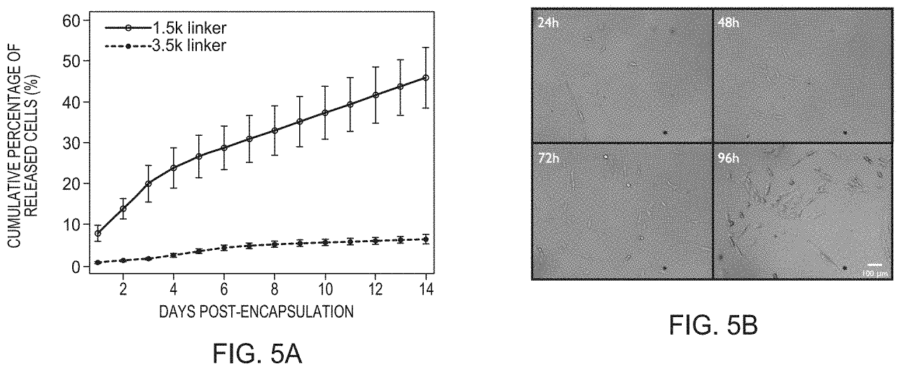

With the 1.5 kDa PEG-dithiol linker, the MSCs had a rapid, initial release immediately after encapsulation/dispersion in the hydrogel. The MSCs were then continuously released over time. The initial burst may be explained by the presence of a big mesh and large holes throughout the hydrogel resulting from gelation defects. With the 3.5 kDa PEG-dithiol linker, however, no burst of cell release was observed throughout the experiment (culturing and incubation was performed as described in the culture/incubation procedure above). Instead, MSCs were released continuously and stably from the hydrogel. Even though the cell release rate was slower compared with the 1.5 kDa PEG-dithiol linker, cell release duration could be significantly prolonged with the larger 3.5 kDa PEG-dithiol linker. After 14 days of culture, about 38% of MSCs were released from the microparticles fabricated with 1.5 kDa PEG-dithiol linker, while only about 7% of cells were released with the 3.5 kDa PEG-dithiol linker as shown in FIG. 5A.

FIG. 5B shows exemplary images illustrating the proliferation of released cells over time (about 96 hours, at intervals of 24 h, 48 h, 72 h, and 96 h). Here, the released MSCs were cultured within 6-well plates after release from the hydrogel. The results show cell proliferation over time, indicating preserved cell functionality. The results also demonstrate that, depending on, for example, clinical timing and dosing needs, variable cell release profiles can be employed with acute or chronic dosing.

### Functionality of Encapsulated MSCs

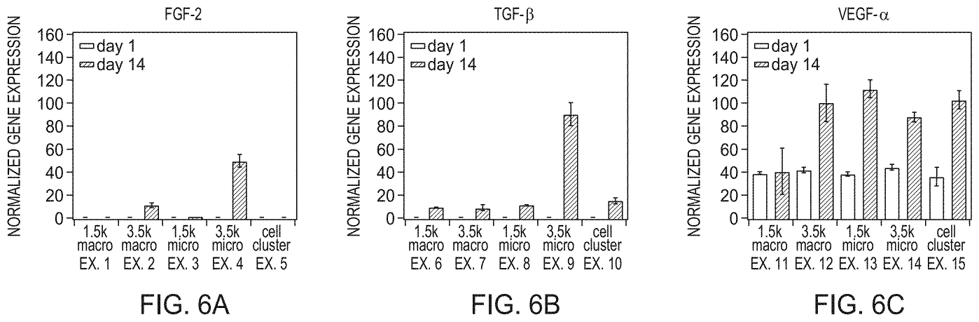

Variability in, for example, hydrogel mesh size and hydrogel stiffness, can result in varied cellular responses. To analyze the effect of microencapsulation/microdispersion and photopolymerization on the functionality of encapsulated/dispersed MSCs, and compare the cellular responses between the two hydrogel properties, the relative expression levels of three target genes—FGF-2, TGF-β, and VEGF-α—were quantitatively measured via qRT-PCR (FIGS. 6A-6C). Expression of these genes were selected based on, for example, potential therapeutic improvements on cartilage repair and regeneration.

MSC-laden macroscopic hydrogels were lysed to compare gene expression between microencapsulation and macroencapsulation. MSC clusters recovered from suspension cultures were analyzed to differentiate the roles that cellular material interactions and cell-cell interactions play in regulating MSC functionality within microparticles. MSC clusters generally refer to clumps of MSCs that form when they are cultured on a surface they cannot attach to. Monolayer MSCs generally refer to the single cells that attach and spread out across a surface as they grow.

To fabricate macro-scale cell-laden hydrogels, MSCs with a density of ˜1×10⁷ cells/mL were mixed with PEGNB, linker and LAP to a final volume of ˜100 with a concentration of ˜10 wt % PEGNB, ˜10 mM linker, and ˜0.1 wt % LAP, within a 1 mL syringe with tip trimmed off. After UV exposure (˜100 mW/cm²) for about 20 seconds, the cell-laden hydrogels were removed and cultured within an ultra-low attachment 6-well plate in the incubator.

FIGS. 6A-6C show exemplary data for the relative (normalized) gene expression of macro- and micro-encapsulated/dispersed MSCs and MSC clusters relative to monolayer MSCs on day 1 and day 14 for the examples in Table 1. Although the monolayer MSCs are not shown, the gene expression of each experimental condition is shown as multiples of the monolayer condition gene expression. Specifically, the gene expression data of FGF-2, TGF-β, and VEGF-α are shown in FIG. 6A, FIG. 6B, and FIG. 6C, respectively. The hydrogels of the MSC-laden microparticles/macroparticles were formed from PEGNB and either the 1.5 kDa PEG-dithiol linker or the 3.5 kDa PEG-dithiol linker. Macroparticles were formed by cutting off the end of a clear syringe, aspirating a cell-containing hydrogel into it, exposing the hydrogel in the syringe to UV light, and ejecting the polymerized cylinder into culture medium. The conditions for the hydrogel and polymerization are given above.

TABLE 1

| | Target | Macroparticle/ | PEG-dithiol linker Utilized |
|---|---|---|---|
| Example | Gene | Microparticle | to Form the Hydrogel |
| Ex. 1 | FGF-2 | Macroparticle | 1.5 kDa PEG-dithiol linker |
| Ex. 2 | FGF-2 | Macroparticle | 3.5 kDa PEG-dithiol linker |
| Ex. 3 | FGF-2 | Microparticle | 1.5 kDa PEG-dithiol linker |
| Ex. 4 | FGF-2 | Microparticle | 3.5 kDa PEG-dithiol linker |
| Ex. 5 | FGF-2 | Cell cluster | |
| Ex. 6 | TGF-β | Macroparticle | 1.5 kDa PEG-dithiol linker |
| Ex. 7 | TGF-β | Macroparticle | 3.5 kDa PEG-dithiol linker |
| Ex. 8 | TGF-β | Microparticle | 1.5 kDa PEG-dithiol linker |
| Ex. 9 | TGF-β | Microparticle | 3.5 kDa PEG-dithiol linker |
| Ex. 10 | TGF-β | Cell cluster | |
| Ex. 11 | VEGF-α | Macroparticle | 1.5 kDa PEG-dithiol linker |
| Ex. 12 | VEGF-α | Macroparticle | 3.5 kDa PEG-dithiol linker |
| Ex. 13 | VEGF-α | Microparticle | 1.5 kDa PEG-dithiol linker |
| Ex. 14 | VEGF-α | Microparticle | 3.5 kDa PEG-dithiol linker |
| Ex. 15 | VEGF-α | Cell cluster | |

Immediately after encapsulation and polymerization, all target genes were expressed from encapsulated MSCs on the same level as to MSCs harvested from monolayer controls. After 14 days in culture, however, encapsulated MSCs had either enhanced or close-to-native expression in selected genes depending on hydrogel property and dimension. Unexpectedly, the elevation in gene expression was hydrogel property- and dimension-dependent. Specifically, MSCs encapsulated with the hydrogel having the 1.5 kDa PEG-dithiol linker had elevated expression in TGF-β, where a 10-fold enhancement was observed within both the macro- and micro-encapsulated cells. This expression pattern was similarly shared with MSC clusters, indicating under this specific condition where diffusional length scale can be omitted, paracrine signaling is as effective as direct cell-cell contact in inducing trophic factor expression. Encapsulation with the 3.5 kDa PEG-dithiol linker resulted in an elevation in FGF-2 and TGF-β expression in both the macro- and micro-encapsulated MSCs. In contrast, microencapsulation had a much more significant enhancement than macroencapsulation, where a ˜50-fold and ˜90-fold improvement was observed for FGF-2 and TGF-β expression in microparticles only. The expression level of genes in macrogels made from the 3.5 kDa PEG-dithiol linker was comparative against MSCs encapsulated with 1.5 kDa PEG-dithiol linker and MSC clusters.

This result may be explained by the presence of differential diffusional length scales of small molecules, such as oxygen and proteins, within the macro- or micro-gel, which can result in hypoxia or starvation-induced cell dysfunction or death. However, the existence of a relatively large mesh size in hydrogels made from the 1.5 kDa PEG-dithiol linker can mitigate this effect by minimizing diffusional length scale variations between macro- and micro-gels. In the case of the macrogels made from the 3.5 kDa PEG-dithiol linker, the increase in scaffold stiffness should stimulate expression of genes in theory as extensively reported. However, and unexpectedly, the MSCs exhibited close-to-native expression pattern over the selected genes.

The gene expression results demonstrated that cellular responses are not altered upon micro- or macro-encapsulation or photopolymerization, but that the cell-material interactions allowed by incubation over time can stimulate the expression of several genes, which could be favorably chosen with high precision via an understanding of cell-material interaction. Cell-laden microparticles, while preserving injectability instead of requiring surgical implantation with macro-scale hydrogels, can also improve cellular functionality. Stimuli, such as cell adhesive motifs, can be further incorporated into this platform for regulating the desired gene expression.

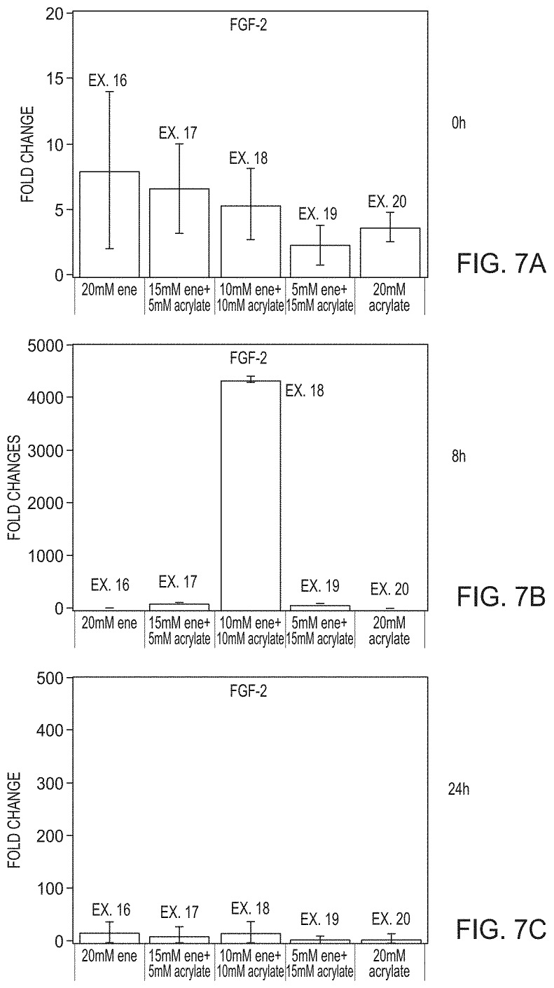

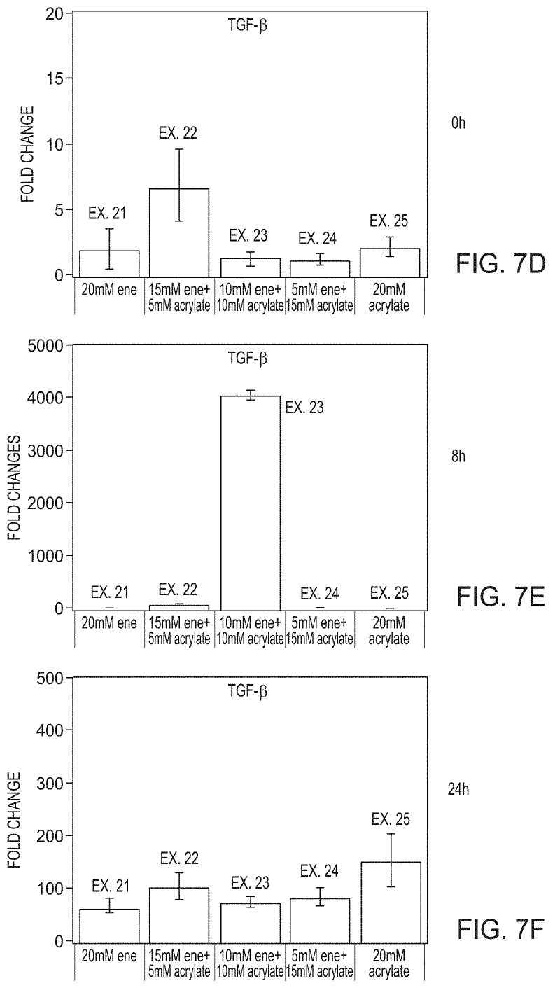

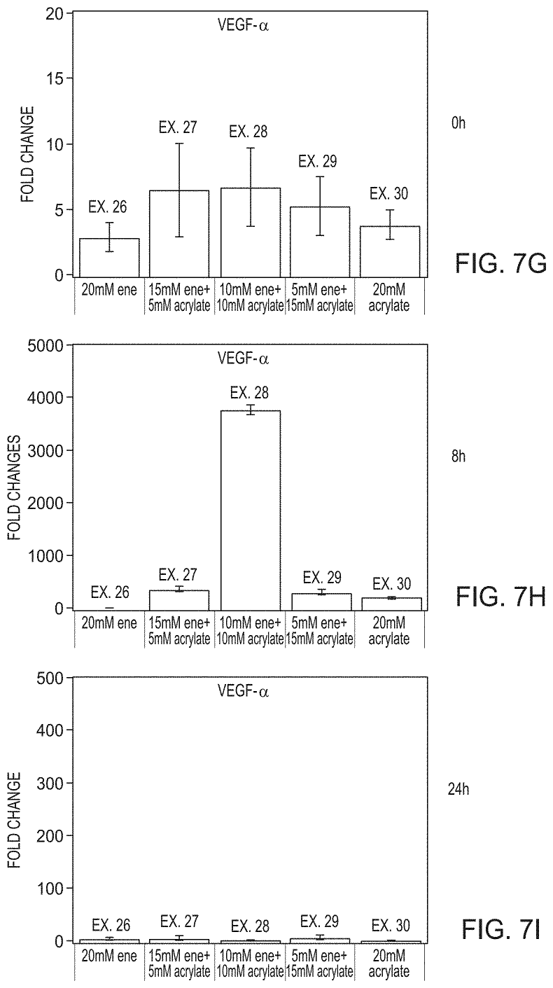

FIGS. 7A-7I show exemplary gene expression data for the examples in Table 2. For the examples in Table 2, the hydrogels were made in the presence of LAP, but without thiol linker. The data illustrate control over cytokine expression of mixed-mode PEG hydrogels—mixtures of PEGNB and PEGDA—and also improved tissue regeneration by tailoring the type and quantity of cytokines produced for a specific application.

TABLE 2

| | Target | Photoreactive Monomers Utilized |
|---|---|---|
| Example | Gene | to Form the Hydrogel |
| Ex. 16 | FGF-2 | 20 mM PEGNB |
| Ex. 17 | FGF-2 | 15 mM PEGNB + 5 mM PEGDA |
| Ex. 18 | FGF-2 | 10 mM PEGNB + 10 mM PEGDA |
| Ex. 19 | FGF-2 | 5 mM PEGNB + 15 mM PEGDA |
| Ex. 20 | FGF-2 | 20 mM PEGDA |
| Ex. 21 | TGF-β | 20 mM PEGNB |
| Ex. 22 | TGF-β | 15 mM PEGNB + 5 mM PEGDA |
| Ex. 23 | TGF-β | 10 mM PEGNB + 10 mM PEGDA |
| Ex. 24 | TGF-β | 5 mM PEGNB + 15 mM PEGDA |
| Ex. 25 | TGF-β | 20 mM PEGDA |
| Ex. 26 | VEGF-α | 20 mM PEGNB |
| Ex. 27 | VEGF-α | 15 mM PEGNB + 5 mM PEGDA |
| Ex. 28 | VEGF-α | 10 mM PEGNB + 10 mM PEGDA |
| Ex. 29 | VEGF-α | 5 mM PEGNB + 15 mM PEGDA |
| Ex. 30 | VEGF-α | 20 mM PEGDA |

### Example Effects of PEG-Dithiol

### E.1. Effect of PEG-Dithiol Linker Length on Crosslinking Kinetics

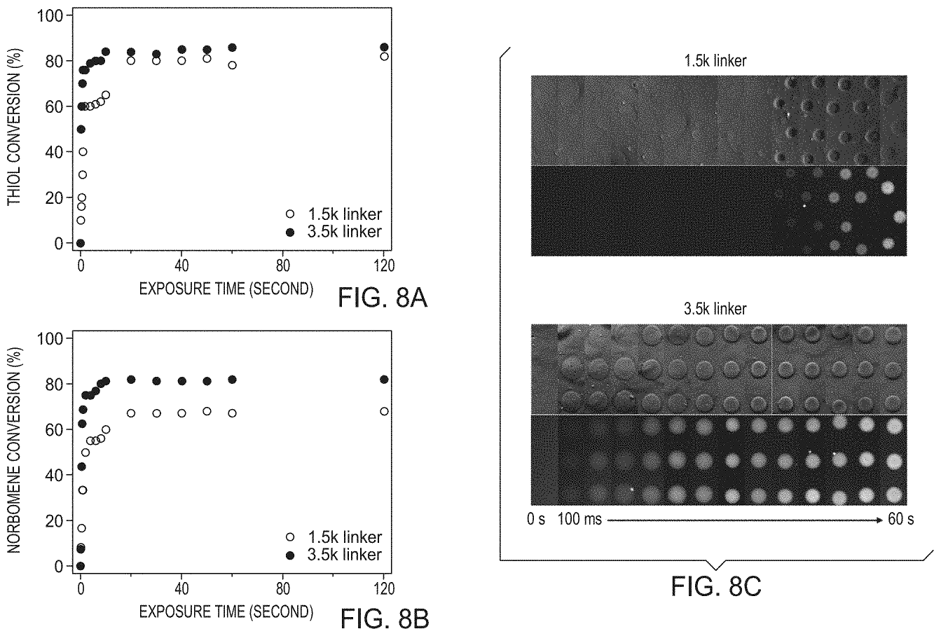

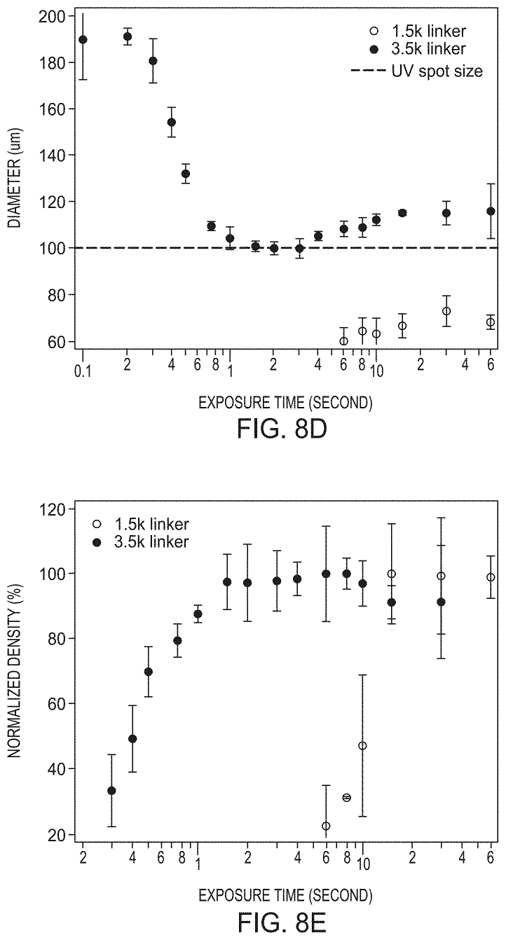

Thiol-ene hydrogels form as a result of crosslinking reactions between multi-arm PEGNB and linear PEG-dithiol molecules. Once a thiol is anchored to an -ene of a particular PEGNB molecule, the opposite thiol can bond with either a different PEGNB molecule to promote network growth, or an -ene from the same PEGNB molecule resulting in linker neutralization. The relative probability of these events occurring depends on the availability and accessibility of -enes in the hydrogel-forming solution. A third reaction is also observed here: the oxidation of thiols to disulfides, which occurs under oxidizing conditions. Hydrogel crosslinking kinetics and hydrogel formation rates were analyzed via thiol and norbornene functional group consumption. As shown in FIG. 8A and FIG. 8B, both thiol and norbornene conversion reached a maximum after about 20 seconds of UV exposure when using the 1.5 kDa PEG-dithiol linker, compared with about 2 seconds of UV exposure when using the 3.5 kDa PEG-dithiol linker.

For these functional group conversion studies, the experiments were conducted with a Raman spectrometer (532 IM-52). To prepare the hydrogel-forming solution, ˜10 wt % PEGNB, ˜10 mM of either 1.5 k PEG dithiol (Sigma-Aldrich, USA) or ˜3.5 k PEG-dithiol (JenKem Technology, USA), and ˜0.1 wt % LAP were mixed thoroughly before polymerization. To prepare the hydrogel samples, ˜30 μl hydrogel-forming solution was polymerized for the following approximate time periods: 0 seconds, 200 milliseconds, 400 milliseconds, 600 milliseconds, 800 milliseconds, 1 second, 2 seconds, 4 seconds, 6 seconds, 8 seconds, 10 seconds, 20 seconds, 30 seconds, 40 seconds, 50 seconds, 60 seconds, and 120 seconds. The thiol conversion was monitored as S—H stretching vibrations at 2570 cm⁻¹. Allyl conversion was monitored as C═C stretching vibrations at 1630 cm⁻¹. Conversions were calculated as the ratio of peak areas of reactant to unreacted solutions prior to polymerization. All reactions were performed under ambient conditions.

FIGS. 8C-8E demonstrate how thiol conversion and norbornene conversion can affect mesh sizes. The thiol conversion efficiency was consistent between the two linkers, while norbornene conversion was dramatically reduced with the 1.5 kDa PEG-dithiol linker. This result indicates that crosslinking occurs faster for 3.5 kDa PEG-dithiol linkers than for 1.5 kDa PEG-dithiol linkers, which is consistent with in situ polymerization studies, in which the hydrogel post size and fluorescence intensity reached their maximums much faster with the 3.5 kDa PEG-dithiol linker (FIGS. 8C, 8D, and 8E). The results indicate that cross-linker length can dictate step-growth hydrogel network formation dynamics and allows rapid on-chip photoencapsulation.

Additionally, the stoichiometric mismatch between thiol and -ene conversion suggests the occurrence of a side reaction that is preferred when using shorter linkers. Indeed, 1H-NMR spectroscopy showed that the rate of disulfide formation with the 1.5 kDa PEG-dithiol linker was significantly higher than the 3.5 kDa PEG-dithiol linker samples. This illustrates that the self-termination of dithiols occurs at a higher frequency with the 1.5 kDa PEG-dithiol linker, which can be directly mapped to the mismatch in thiol and -ene conversion efficiency with 1.5 kDa PEG-dithiol linker.

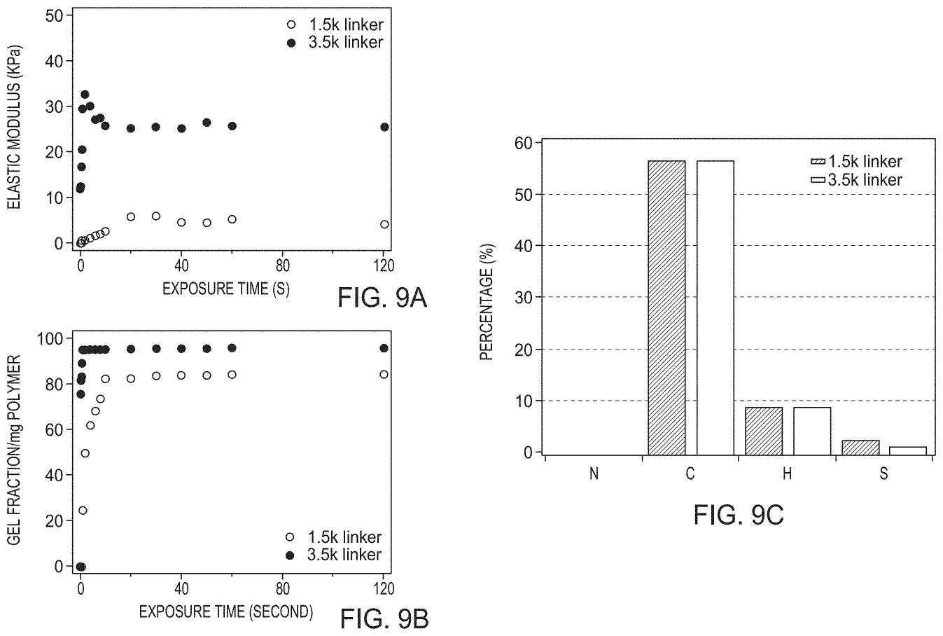

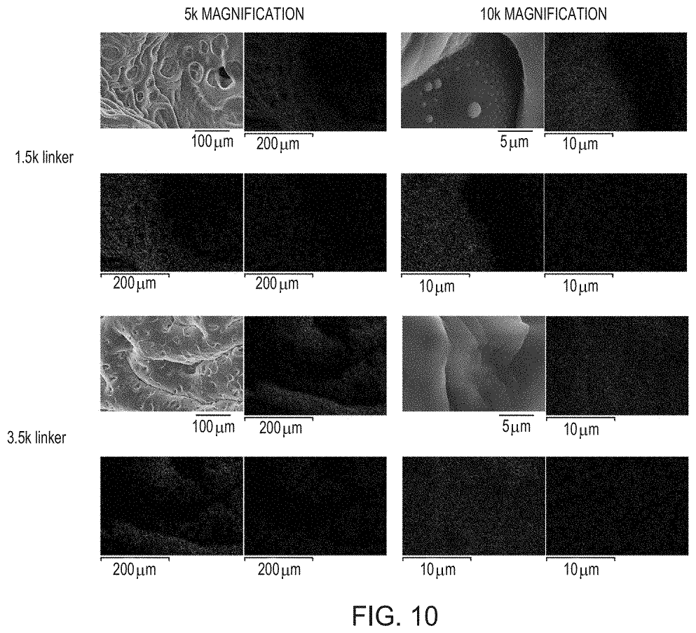

To further investigate the gelation efficiency, the elastic modulus and gel fraction per mg polymer was measured. Results are shown in FIG. 9A and FIG. 9B. With the 1.5 kDa PEG-dithiol linker, hydrogels had an increase in stiffness as the UV exposure time increased to about 20 seconds, at which point a plateau was reached. As with the 3.5 kDa PEG-dithiol linker, the maximum stiffness was rapidly reached after about 2 seconds of UV exposure (FIG. 9A). The extent of gelation reached a peak after about 20 seconds of UV exposure with the 1.5 kDa PEG-dithiol linker, while it reached its maximum within about 2 seconds with the 3.5 kDa PEG-dithiol linker, following the trend observed in the mechanical testing, functional group conversion, and in situ polymerization studies described above (FIG. 9B). About 20 wt % of the macromers could not be incorporated into the hydrogel network under any circumstances with the 1.5 kDa PEG-dithiol linker. Combined with the decreased norbornene conversion efficiency and loss in the observed hydrogel mechanical strength, this likely indicates that the mass loss of unreacted or neutralized ene-containing PEGNB accounts for the overall loss of hydrogel mass.

FIG. 9C shows elemental analysis for the example hydrogels made with the 1.5 kDa PEG-dithiol linker or the 3.5 kDa PEG-dithiol linker. Here, the percentage of carbon (C) and hydrogen (H) in hydrogels formed with the two linkers was comparable; however, the observed sulfur (S) fraction (mass S/total hydrogel mass) in hydrogels formed with the 1.5 kDa PEG-dithiol linker was two-fold that of the S fraction in hydrogels formed with the 3.5 kDa PEG-dithiol linker. It is believed that shorter linkers either undergo linker neutralization or otherwise fail to crosslink PEGNB at a higher rate than longer linkers. While the relative fraction of S in the formed PEGNB hydrogels varied significantly with the linker length, the total S mass was similar for both samples. This indicates that thiol conversion was similar for both linkers and the difference in S fraction was due to the systematic mass loss of PEGNB that had not been incorporated into the network (FIG. 9C). Under identical experimental conditions, the resulting reduced hydrogel stiffness, norbornene conversion efficiency, and gelation efficiency with the 1.5 kDa PEG-dithiol linker could be explained by the possibility that linker neutralization and self-termination occurred more frequently with the 1.5 kDa PEG-dithiol linker, resulting in the loss of unreacted -enes. The 3.5 kDa PEG-dithiol linker, conversely, improved crosslinking kinetics and gelation efficiency, thus producing increased elastic modulus, presumably because elongated linkers have a lower probability of self-terminating and an increased possibility of anchored crosslinkers between discrete macromers, thus promoting network growth. Finally, an additional contributor to the network formation is the promotion of entanglements by longer linkers. Moreover, supplementation with excess 1.5 kDa PEG-dithiol linker did not improve gelation efficiency, further indicating that linker neutralization and self-termination occurs simultaneously within the system.

### E.2. Effect of PEG-Dithiol Linker Length on Formed Hydrogel Properties

To further validate that linker neutralization and self-termination occur more frequently with a short linker and to characterize hydrogel properties, polymerized hydrogels were imaged by SEM as shown in FIG. 10. The presence of carbon, oxygen, and sulfur throughout the hydrogel is indicated as red, green, and blue, respectively. Large pores only presented in hydrogels made with the 1.5 kDa PEG-dithiol linker. Additionally, polymer debris or pieces were observed in the large pores under 10 k magnification, indicating a lack of connectivity to form uniform networks with the 1.5 kDa PEG-dithiol linker. In gels formed with the 3.5 kDa PEG-dithiol linker, however, crosslinking and gelation efficiency were significantly improved as a result of the increased availability of unreacted thiols, and there was a higher probability of PEG-dithiol crosslinking different PEGNB molecules due to the linkers' greater persistence length. In addition, the presence of ester bonds made the PEGNB hydrogels susceptible to hydrolysis.

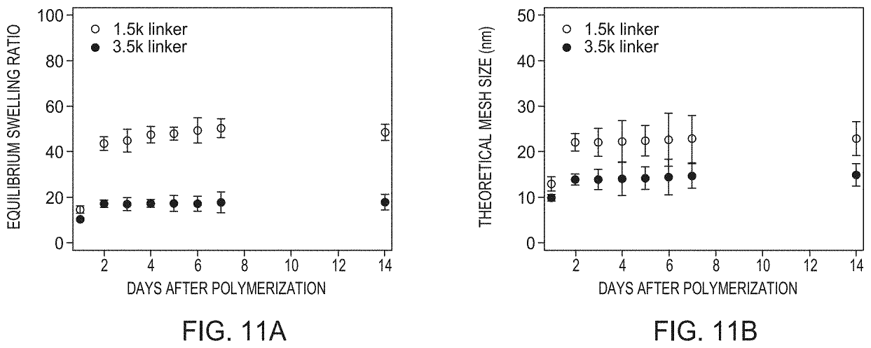

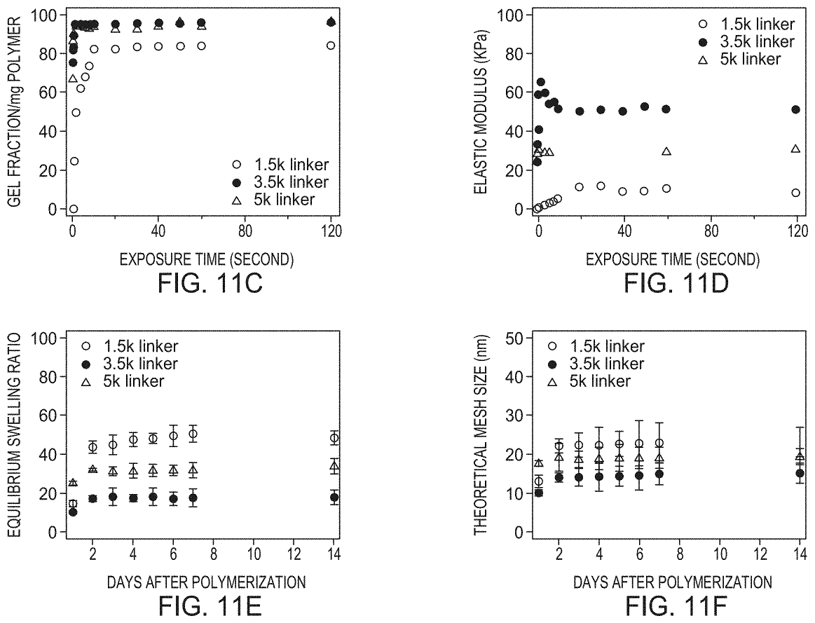

Hydrogels made with the 1.5 kDa PEG-dithiol linker showed an increased rate of increase in the swelling ratio, and thus the theoretical mesh size over time, while those hydrogels made with the 3.5 kDa PEG-dithiol linker had a smaller mesh size initially, and only increased slightly within the given time frame (FIG. 11A and FIG. 11B). Hydrogels made with the 1.5 kDa PEG-dithiol linker showed comparable gelation efficiency to the 3.5 kDa PEG-dithiol linker, but their mesh size and mechanical strength (elastic modulus) fell between that of the 1.5 kDa PEG-dithiol linker and the 3.5 kDa PEG-dithiol linker (FIGS. 11C-11F), indicating that the macromolecular properties contribute more strongly to the gel properties once the gelation efficiency surpasses a threshold. The formed hydrogel properties would thus positively correlate with linker length. Also shown in FIGS. 11C-11F are data for hydrogels made with a 5 kDa PEG-dithiol linker.

### E.3. Tuning Formed Hydrogel Dynamics Via Manipulating Linker Composition

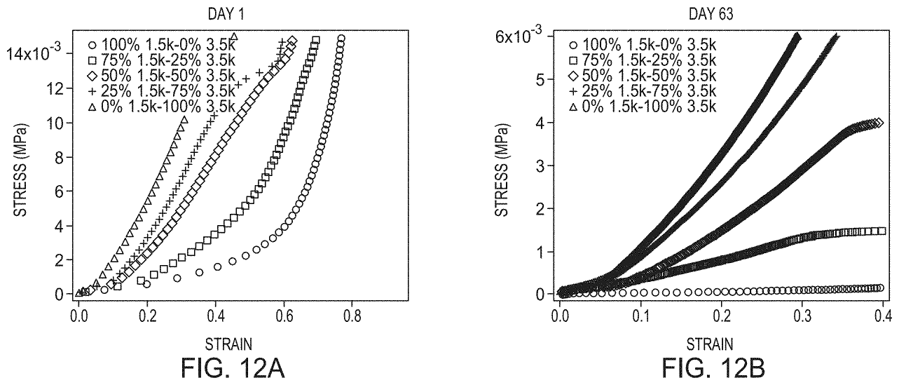

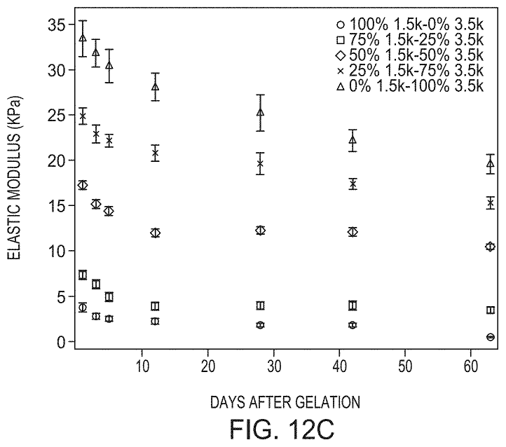

By combining the two linkers according to a fixed molar gradient, the formed hydrogel properties may be tuned. The mechanical strength of the hydrogel samples behaved in a 3.5 kDa PEG-dithiol linker concentration-dependent manner, where improvement of mechanical properties was observed as more 3.5 kDa PEG-dithiol linker was utilized (FIG. 12A). Over 63 days of incubation, all hydrogels partially degraded, which was reflected in a decreased modulus for all samples. However, the mechanical properties of hydrogels with varying linker compositions were still strikingly different (FIG. 12B and FIG. 12C), indicating that network ideality increases with an increase in the proportion of 3.5 kDa PEG-dithiol linker. This result suggests that the degradation behavior and structural integrity of PEGNB hydrogels can be favorably tuned and customized to fulfill the needs of a variety of treatment gaps.

It was found that by varying the linker length, hydrogel-forming rates could be favorably tuned. Meanwhile, the formed hydrogels were found to have different physical properties even after reaching maximum conversion, which could be attributable to the variable gelation efficiency and network ideality results from the availability of -enes to anchored thiols. By proportionally mixing 1.5 kDa and 3.5 kDa PEG-dithiol linkers while keeping the total thiol molar concentration constant, the formed PEGNB hydrogel properties could be favorably tuned in a linear relationship to the proportion of the 3.5 kDa PEG-dithiol linker. To take advantage of this property control, and also fulfill the demand for the rapid reaction rates utilized to fabricate hydrogel features on-chip with droplet microfluidics, the 3.5 kDa PEG-dithiol linker with improved crosslinking kinetics and network ideality was successfully utilized to fabricate PEGNB microgel slugs, discs, and spheres on-chip by manipulating relative flow rates within a defined channel geometry and dimension. Moreover, to validate the translational potential and cytocompatibility of this platform, 3T3 fibroblasts were encapsulated and polymerized into PEGNB microgel features on-chip. Over about 90% of the encapsulated cells remained viable after culturing for 14 days, indicating that this platform has minimal toxicity to cells. This understanding of thiol-ene chemistry, combined with high-throughput droplet microfluidics, offers a versatile one-step microgel fabrication platform to rapidly fabricate microgels with excellent tunability in hydrogel dynamics, and provides an alternative route for tissue engineering and regenerative medicine for cell-based therapeutics.

Various approaches to the on-chip fabrication of cell-laden hydrogel particles on microlength scales are described herein. These hydrogel particles are formed by, for example, thiol-ene based chemistry between an -ene group of a polymerizable monomer (for example, PEGNB, PEGDA, etc.) and a PEG-dithiol linker in a microfluidic device. After 14 days of culture of the hydrogel-encapsulated/dispersed cells, the cells maintained cell viability and functionality for stimulating cartilage repair as the cells are slowly released from the hydrogels. Adjusting hydrogel properties by the type of linker, polymerizable monomer, and polymerization conditions, among other parameters, results in various cell release profiles and cellular responses. For example, close-to-native gene expression and/or significantly upregulated gene expression can be achieved by varying hydrogel properties and dimensions. This cytocompatible cell encapsulation/dispersion platform offers an alternative route for tissue engineering and to surgery-free cell therapies.

In the foregoing, reference is made to embodiments of the disclosure. However, it should be understood that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the foregoing aspects, features, embodiments and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

As is apparent from the foregoing general description and the specific aspects, while forms of the aspects have been illustrated and described, various modifications can be made without departing from the spirit and scope of the present disclosure. Accordingly, it is not intended that the present disclosure be limited thereby. Likewise, the term “comprising” is considered synonymous with the term “including.” Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “Is” preceding the recitation of the composition, element, or elements and vice versa, such as the terms “comprising,” “consisting essentially of,” “consisting of” also include the product of the combinations of elements listed after the term.

For purposes of this present disclosure, and unless otherwise specified, the term “coupled” is used herein to refer to elements that are either directly connected or connected through one or more intervening elements. For example, an opening can be directly connected to the fluidic channel, or it can be connected to the fluidic channel via intervening elements.

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. For example, the recitation of the numerical range 1 to 5 includes the subranges 1 to 4, 1.5 to 4.5, 1 to 2, among other subranges. As another example, the recitation of the numerical ranges 1 to 5, such as 2 to 4, includes the subranges 1 to 4 and 2 to 5, among other subranges. Additionally, within a range includes every point or individual value between its end points even though not explicitly recited. For example, the recitation of the numerical range 1 to 5 includes the numbers 1, 1.5, 2, 2.75, 3, 3.80, 4, 5, among other numbers. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise. For example, aspects comprising “a monomer” include aspects comprising one, two, or more monomers, unless specified to the contrary or the context clearly indicates only one monomer is included.

While the foregoing is directed to embodiments of the present disclosure, other and further embodiments of the disclosure may be devised without departing from the basic scope thereof, and the scope thereof is determined by the claims that follow.

What is claimed is:

1. A composition for controlling production of secretomes, the composition comprising:
a hydrogel comprising, in polymerized form, one or more photoreactive monomers and a thiol linker, wherein at least one of the one or more photoreactive monomers comprises a methylene functional group, an acid functional group, or combinations thereof; and
one or more cells dispersed or encapsulated within the hydrogel.

2. The composition of claim 1, wherein less than about 20% of the cells are released from the hydrogel within 48 hours after encapsulation or dispersion within the hydrogel.

3. The composition of claim 1, wherein a diameter of the hydrogel is from about 100 μm to about 180 μm.

4. The composition of claim 1, wherein about 80% or more of the cells remain viable for 300 hours or more after dispersion or encapsulation within the hydrogel.

5. The composition of claim 1, wherein the one or more photoreactive monomers comprises polyethylene glycol norbornene, polyethylene glycol diacrylate, polylactic acid, derivatives thereof, or combinations thereof.

6. The composition of claim 1, wherein the thiol linker has a molecular weight of about 10 kDa or less.

7. The composition of claim 1, wherein the thiol linker has a molecular weight of about 5 kDa or less.

8. A composition for controlling cytokine production, comprising the composition of claim 1.

9. A controlled release composition, comprising the composition of claim 1.

10. A method of forming a composition for controlling production of secretomes, comprising:
introducing a cell to one or more photoreactive monomers and a thiol linker in a microfluidic device to form a reaction mixture; and
polymerizing the reaction mixture by exposure to ultraviolet light, under polymerization conditions, to form the composition for controlling production of secretomes.

11. The method of claim 10, wherein the polymerization conditions comprise:
a duration of exposure to ultraviolet light from about 1 millisecond to about 60 seconds;
an energy density of the ultraviolet light from about 1 mW/cm² to about 10,000 mW/cm²;
a pH of the reaction mixture from about 5 to about 9; or
a combination thereof.

12. The method of claim 10, wherein the composition is in the form of one or more cells dispersed or encapsulated within a hydrogel.

13. The method of claim 12, wherein the hydrogel is formed from the one or more photoreactive monomers and the thiol linker.

14. The method of claim 13, wherein the one or more photoreactive monomers comprise polyethylene glycol norbornene, polyethylene glycol diacrylate, polylactic acid, derivatives thereof, or a combination thereof.

15. The method of claim 13, wherein the thiol linker has a molecular weight of about 5 kDa or less.

16. The method of claim 13, wherein the one or more photoreactive monomers comprise polyethylene glycol norbornene.

17. The method of claim 12, wherein a diameter of the hydrogel is from about 100 μm to about 180 μm.

18. A method for forming a therapeutic dose of a composition for controlling production of secretomes, comprising:
introducing a cell to one or more components to form a reaction mixture;
polymerizing the reaction mixture, under polymerization conditions, to form a cell dispersed or encapsulated within a hydrogel, the hydrogel comprising, in polymerized form, one or more photoreactive monomers and a thiol linker;
purifying, under purification conditions, the cell dispersed or encapsulated within the hydrogel to form a purified hydrogel-encapsulated cell and/or hydrogel-dispersed cell; and
suspending the purified hydrogel-encapsulated cell and/or hydrogel-dispersed cell in a formulation to form the therapeutic dose of the composition.

19. The method of claim 18, wherein:
the one or more photoreactive monomers comprises polyethylene glycol norbornene, polyethylene glycol diacrylate, polylactic acid, derivatives thereof, or a combination thereof;
the thiol linker has a molecular weight of about 10 kDa or less; or
combinations thereof.

20. The method of claim 18, wherein a diameter of the hydrogel is from about 100 μm to about 180 μm.
